(19) United States
(12) Patent Application Publication
(10) Pub. No.: US 2006/0069124 A1
(43) Pub. Date: Mar. 30, 2006

(54) Use of MDL-100,907 for treatment of allergic and eosinophil mediated diseases

(76) Inventors: P. Srirama Rao, San Diego, CA (US); Stefan Boehme, Encinitas, CA (US); Savita P. Rao, San Diego, CA (US); Goukanapalli Chandrasekara Reddy, Bangalore (IN)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 11/221,668

(22) Filed: Sep. 7, 2005

Related U.S. Application Data
(60) Provisional application No. 60/607,886, filed on Sep. 7, 2004.

(51) Int. Cl. A61K 31/445

(52) U.S. Cl. 514/317

(57) ABSTRACT

Methods of modulating eosinophil migration, chemotaxis or generation, in vitro, ex vivo, and in vivo are provided. Methods include contacting eosinophils with an amount of 5-HT2A receptor agonist or antagonist sufficient to modulate eosinophil migration, chemotaxis or generation.

### RELATED APPLICATIONS

This application claims priority to application Ser. No. 60/607,886, filed Sep. 7, 2004, which application is incorporated by reference herein in its entirety.

### STATEMENT AS TO FEDERALLY SPONSORED RESEARCH

The invention was supported in part by National Institute of Health RO1 Grants AI35796 and HL079304. The government may therefore have certain rights in the invention.

### INTRODUCTION

Eosinophils are of the granulocyte lineage of hematopoetic cells that are potent proinflammatory cells. A predominant physiological role for eosinophils is to rid the body of helmenthic infections, and this is accomplished in part by their powerful arsenal of secretory granule proteins and release of inflammatory mediators. Eosinophils normally migrate from the bone marrow to the intestinal lumen (e.g. duodenum) via the bloodstream. This process is tightly regulated as IL-5 plays a major role in governing eosinophil maturation and release from the bone, marrow. Chemoattractants, such as chemokines (e.g. eotaxin), play a critical role in recruiting eosinophils from the bloodstream to tissues by a highly orchestrated process characterized by eosinophil rolling along the vessel wall, firm adhesion and extravasation into the target tissue.

However, dysregulated trafficking and degranulation of eosinophils has been shown to play a critical role in the pathophysiology of a number of human diseases. In various disease states, normal eosinophil trafficking in the body is disrupted by inappropriate or dysregulated expression of eosinophil chemoattractants. Thus, identifying the various chemoattractants and their cognate receptors expressed by eosinophils open a potentially powerful therapeutic avenue to treat diseases caused in part by aberrant or undesirable eosinophil recruitment to tissues and subsequent release of proinflammatory mediators and ultimately degranulation. Hence, identification and antagonism of eosinophil chemoattractants, particularly those involved in human disease, has great therapeutic value.

In the human disease of allergic asthma, accumulation of eosinophils in the airways has been noted in clinical, post-mortem analysis as well as in experimental animal models of asthma and allergic inflammation (DeMonchy, et al. American Review of Respiratory Disease 131:373 (1985); Durham, et al. Clinical Allergy 15:411 (1985)). Increased number of circulating eosinophils are observed not only in the airways of patients with bronchial inflammation and allergen-induced late phase responses, but are prominently present in the sputum and lavage fluids of patients with asthma (Bousquet, et al. New England Journal of Medicine 323:1033 (1990); Jeffery, et al. American Review of Respiratory Diseases 140:1745 (1989)). Activated eosinophils play an inflammatory role by generating lipid mediators such PAF and LTC4, as well as cytokines and toxic granule proteins (Briskin, et al. Nature 363:461 (1993); Broide, et al. J Allergy Clin Immunol 89:958 (1992); Broide, et al. Immunol. Reviews 179:163 (2001); Weller, et al. Eur Respir J Suppl 22: 109s (1996)). The increase in eosinophils is usually associated with severity of the disease and overall these observations provide substantial evidence that eosinophils have a causative role in pathogenesis of allergic inflammation and respiratory tissue damage.

Serotonin (5-HT) is one of the most extensively studied neurotransmitters of the central nervous system. It is also present in a variety of peripheral tissues including in constituents of the immune system. 5-HT is known to play a role in T cell and natural killer cell activation, delayed-type hypersensitivity responses and production of chemotactic factors (Mossner, et al. Brain Behav Immun 12:249 (1998)). In addition to its function as a neurotransmitter, 5-HT is, released by mast cells and may play a role in the pathophysiology of asthma (Nomura, et al. J Lab Clin Med 138:226 (2001)). Increased levels of free 5-HT have been shown to be present in the plasma of symptomatic asthmatic patients compared to asymptomatic patients (Cazzola et al. Trends Pharmacol Sci. 21:13.(2000); Cazzola, et al. Allergy 50:1 (1995)).

Serotonin has been shown to have at least seventeen distinct receptors (Barnes, et al. Neuropharmacology 38:1083 (1999)). 5-HT receptors are known to include both ion-channel type receptors and G-coupled protein linked receptors (GPCRs). The 5-HT2 subclass of receptors (2A, 2B, 2C) are GPCRs that, until recently, were difficult to distinguish pharmacologically due to a lack of receptor specific inhibitors. MDL-100,907 is a highly selective 5-HT2A antagonist (Kehne, et al. Neuropsychopharmacology 15:116 (1996); U.S. Pat. No. 5,134,149).

### SUMMARY

The invention is based at least in part on the finding that serotonin (5-HT) is a potent eosinophil chemoattractant whose effect is mediated by 5-HT2A receptor. The invention is also based upon the finding that antagonists targeting the 5-HT2A receptor, including the 5-HT2A receptor specific antagonist MDL-100,907, can inhibit serotonin-mediated eosinophil chemotaxis in vitro. Additionally, in an animal model of allergic inflammation (airway hyperresponsiveness-hyperreactivity (AHR)) using an aerosolized antigen, pulmonary eosinophilia and concomitant AHR can be inhibited by intraperitoneal injection of MDL-100,907. This blockade of pathologic eosinophil recruitment by a specific 5-HT2A receptor antagonist coupled with the in vitro activity observed with human eosinophils indicates that other eosinophil-mediated allergic or disease states, including the generation of eosinophils in bone marrow, can be treated by administration of 5-HT2A receptor antagonists including 4-piperidine-methanol and N-aralkyl-piperidine-methanol derivatives, in particular, for example, (+)-α-(2,3-dimethoxyphenyl)-1-[2-(4-fluorophenyl)ethyl]-4-piperidinemethanol (MDL-100,907); α-(3,4-dimethoxypheriyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, racemate (+ or − enantiomer) or prodrug thereof salts, free bases, esters, derivatives, racemates (+ or − enantiomers) and prodrugs thereof, and the like. The favorable safety profile of MDL-100,907 in both preclinical animal studies and human clinical trials indicate that this compound is a safe and effective therapeutic for a variety of human diseases when either used alone or in combination with existing therapies.

In accordance with the invention, there are provided, methods of modulating eosinophil migration, chemotaxis or generation. In one embodiment, a method includes contacting eosinophils with an amount of 5-HT2A receptor agonist or antagonist sufficient to modulate eosinophil migration, chemotaxis or generation. In various aspects, migration, chemotaxis or generation is reduced, decreased, inhibited, delayed, or prevented; or increased, stimulated, enhanced, promoted or induced. 5-HT2A receptor agonists and antagonists can be selective for 5-HT2A receptor. In particular aspects, a 5-HT2A receptor antagonist includes a 4-piperidine-methanol or N-aralkyl-piperidine-methanol derivative. In more particular aspects, antagonists include (+)-α-(2,3-dimethoxyphenyl)-1-[2-(4-fluorophenyl)ethyl]-4-piperidine methanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer) or prodrug thereof.

Invention methods of modulating eosinophil migration, chemotaxis or generation include contact or administration, in vitro or in vivo (e.g., to a subject in need of modulating eosinophil migration, chemotaxis or generation). In one embodiment, eosinophil migration, chemotaxis or generation is reduced, decreased, inhibited, delayed, halted, or prevented locally, or regionally in a tissue or organ of a subject. In one aspect, eosinophil migration, chemotaxis or generation is reduced, decreased, inhibited, delayed, halted, or prevented in a pulmonary tissue or organ, gut, or bone marrow. In another aspect, eosinophil migration, chemotaxis or generation is reduced, decreased, inhibited delayed, halted, or prevented in lung, airways or respiratory mucosum. In yet another embodiment, the in vivo contacting is in a subject that has previously experienced an asthmatic episode or airway- or broncho-constriction or is in need of airway- or broncho-dilation.

In accordance with the invention, there are also provided, methods of reducing or decreasing progression, severity, frequency, duration or probability of one or more symptoms associated with asthma. In one embodiment, a method includes administering to a subject an amount of 5-HT2A receptor antagonist sufficient to reduce or decrease progression, severity, frequency, duration or probability of a symptom associated with asthma. In various aspects, the asthma is caused by an allergen or by exercise. In additional aspects, the symptom is selected from lung, airways or respiratory mucosum inflammation or tissue damage, shortness of breath, wheezing, coughing, chest-tightness, chest pain, increased heart rate, runny nose, airway-constriction, decreased lung capacity, or an acute asthmatic episode.

In accordance with the invention, further provided are methods of treating a subject having or at risk of having a condition associated with undesirable or abnormal eosinophil migration, chemotaxis or generation. In one embodiment, a method includes administering to a subject an amount of 5-HT2A receptor antagonist sufficient to reduce or decrease undesirable or abnormal eosinophil migration, chemotaxis or generation thereby treating the subject. In various aspects, the condition includes a chronic or acute allergic disorder (e.g. Extrinsic bronchial asthma; Allergic rhinitis; Onchocercal dermatitis; Atopic dermatitis, Drug reactions; Nodules, eosinophilia, rheumatism, dermatitis, and swelling (NERDS); Eosophageal or GI allergies). In additional aspects, the condition includes a vasculitic granulomatous disease (e.g., Temporal vasculitis; Churg-Strauss syndrome; Polyarteritis; Wegener's granulomatosis; Eosinophilic granulomatous prostatitis; or Ulercerative colitis), an immunological disorder, such as an autoimmune disease (e.g., multiple sclerosis), graft rejection or Intrinsic bronchial asthma. In further aspects, the condition includes an interstitial disorder or a pulmonary disorder, such as Eosinophilic pleural effusions; Transient pulmonary eosinophilic infiltrates (Löffler); Histiocytosis; Chronic eosinophilic pneumonia; Hypersensitivity pneumonitis; Allergic bronchopulmonary aspergillosis; Sarcoidosis; Idiopathic pulmonary fibrosis; pulmonary edema; pulmonary embolism; pulmonary emphysema; Pulmonary Hyperventilation; Pulmonary Alveolar Proteinosis; Chronic Obstructive Pulmonary Disease; Interstitial Lung Diseases; or Topical eosinophilia. In still further aspects, the condition includes a respiratory disorder or a respiratory mucosum disorder, such as Airway Obstruction, Apnea, Asbestosis, Atelectasis, Berylliosis, Bronchiectasis, Bronchiolitis, Bronchiolitis Obliterans Organizing Pneumonia, Bronchitis, Bronchopulmonary Dysplasia, Common Cold, Cough, Empyema, Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Respiratory Tract Infections, Rhinoscleroma, Scimitar Syndrome, Severe Acute Respiratory Syndrome, Silicosis, Tracheal Stenosis or Whooping Cough. In yet additional aspects, the condition includes a neoplastic or myeloproliferative disease (e.g., Hypereosinophilic syndrome).

In another embodiment, a method includes an amount administered sufficient to reduce or decrease progression, severity, frequency, probability, duration or prevent one or more adverse physiological or psychological symptoms associated with a condition, disorder or disease associated with undesirable or abnormal eosinophil migration, chemotaxis or generation. In particular aspects, a condition, disorder or disease is allergic asthma, or an acute asthmatic episode.

Invention treatment methods include providing a subject with an objective or subjective improvement of the condition, disorder or disease, a symptom associated with the condition, disorder or disease, or the probability or susceptibility of a subject to the condition or a symptom associated with the condition, disorder or disease. In various embodiments, treatment reduces, decreases, inhibits, delays, eliminates or prevents the probability, susceptibility, severity, frequency, or duration of one or more symptoms associated with or caused by the condition, disorder or disease. In a particular aspect, a method reduces or decreases the probability, severity, frequency, duration or preventing a subject from having an acute asthmatic episode (e.g., an acute asthmatic episode caused by allergic asthma). In another particular aspect, a method reduces the probability, severity, frequency, duration or delays, halts, or prevents airway-constriction. In various embodiments, treatment improves the condition, disorder or disease. In a particular aspect, a method increases airway-dilation.

Method of the invention can be practiced using selective or non-selective agonists or antagonists. In one embodiment, the agonist or antagonist binds to 5-HT2A receptor. In particular aspects, a method of the invention includes an antagonist, for example, a 4-piperidine-methanol or N-aralkyl-piperidine-methanol derivative. Such methods include, for example, (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol, α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifouorethoxyphenyl)ethyl]-4-piperidine methanol, or a salt, free base, ester, derivative, racemate (+ or − enantiomer) or prodrug thereof.

Candidate subjects for methods of the invention include mammals, such as humans. Candidate subjects for methods of the invention further include subjects that have or are at risk of having a condition, disorder or disease associated with undesirable or abnormal eosinophil migration, chemotaxis or generation. In particular aspects, a subject has been diagnosed as having asthma or is at risk of having asthma.

Methods of the invention can be practiced using dose amounts, frequencies, delivery routes and timing of agonist or antagonist sufficient or effective for the intended purpose. In particular embodiments, a subject is administered agonist or antagonist one, two, three, four or more times daily, weekly, monthly or annually. In additional embodiments, the amount administered is about 0.00001 mg/kg, to about 10,000 mg/kg, about 0.0001 mg/kg, to about 1000 mg/kg, about 0.001 mg/kg, to about 100 mg/kg, about 0.01 mg/kg, to about 10 mg/kg, about 0.1 mg/kg, to about 1 mg/kg one, two, three, four, or more times per hour, day, week, month or annually. In further embodiments, the amount administered is less than about 0.00001 mg/kg, one, two, three, four, or more times per hour, day, week, month or annually. In particular aspects, the amount is administered substantially contemporaneously with, or within about 1-60 minutes, hours, or days of the onset of a symptom associated with undesirable or abnormal eosinophil migration, chemotaxis or generation (e.g., allergic. asthma, an asthmatic episode or airway-constriction).

Methods of the invention include routes of contact or administration of agonist or antagonist systemically, locally or regionally. In a particular embodiment, a 5-HT2A receptor agonist or antagonist is delivered to lungs or airways.

Methods of the invention can be practiced in conjunction with one or more other treatment protocols or therapeutic regimens. In a particular embodiment, a method includes contacting or administering a second drug to the subject prior to, with or following contacting or administering a 5-HT2A receptor agonist or antagonist. In particular aspects, a second drug includes an anti-inflammatory, anti-asthmatic or anti-allergy drug; a hormone or a steroid; an anti-histamine, anti-leukotriene, anti-IgE, anti-α4 integrin, anti-β2 integrin, anti-CCR3 antagonist, β2 agonist or an anti-selectin.

Methods of the invention may optionally exclude subjects previously administered a 5-HT2A receptor antagonist (e.g., 4-piperidine-methanol or N-aralkyl-piperidine-methanol derivative, such as, (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer) or prodrug thereof), also having a condition, disorder or disease associated with undesirable or abnormal eosinophil migration, chemotaxis or generation, where the amount administered to the subject modulates eosinophil migration, chemotaxis or generation, for example. In particular aspects, an excluded subject has previously been administered a 4-piperidine-methanol or N-aralkyl-piperidine-methanol derivative for treatment of an acute or chronic neurological or psychological disorder. Exemplary neurological or psychological disorders include anxiety, anorexia nervosa, insomnia, sleep apnea, obsessive compulsive disorder, psychosis (e.g., brief, shared or substance-induced delusions, hallucinations, or illusions), bipolar disorder, depression, dysthymia, schiozophrenia, mania, substance abuse (e.g., chronic or acute alcohol, nicotine, a narcotic, an opiate, a stimulant, cocaine, amphetamine, methamphetamine or dextroamphetamine abuse), or migraines. In additional aspects, an excluded subject has previously been administered a 4-piperidine-methanol or N-aralkyl-piperidine-methanol derivative for treatment of an acute or chronic cardiac disorder (e.g., myocardial infarction, ischemia, stable or variant angina, coronary vasospasms, or coronary arrhythmia, such as atrial tachycardia, atrial flutter, atrial fibrillation, ventricular tachycardia or ventricular fibrillation), vascular disorder (e.g., peripheral vascular disease, glaucoma, intermittent claudication, peripheral vasospasms, a thrombotic illness, an embolitic illness or stroke) or hypertension. In further aspects, an excluded subject has previously been administered a 4-piperidine-methanol or N-aralkyl-piperidine-methanol derivative for treatment of fibromyalgia or Raynaud's phenomenon; or as an anesthetic or analgesic. In still further aspects, an excluded subject has previously been administered a 4-piperidine-methanol or N-aralkyl-piperidine-methanol derivative for treatment of an acute or chronic extrapyramidal side effect (EPSE) associated with a neuroleptic drug, such as dysphoria, akathisia, a cognitive impairment, parkinsonian-like syndrome, tremors, or loss of motivation.

Invention compositions can be formulated as appropriate for practice of the methods. In one embodiment, a composition includes an agonist or antagonist, and a pharmaceutically acceptable carrier. In a particular aspect, the carrier is a physiologically acceptable gas or liquid (e.g., an aerosol or dry powder). In an additional particular aspect, the carrier is a capable of traversing into epithelium of a mucosal tissue. In another particular aspect, the carrier is substantially incapable of traversing the blood-brain or blood-spinal cord barrier. In a further particular aspect, the carrier is non-lipophilic. In yet additional particular aspects, the carrier does not traverse the blood-brain or blood-spinal cord barrier of the subject in sufficient amounts effective to treat a subject suffering from an acute or chronic neurological or psychological disorder.

Invention compositions can also be included in articles of manufacture or kits appropriate for practice of the invention methods. In one embodiment, a 5-HT2A receptor agonist or antagonist (e.g., (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethhyl]-4-piperidine methanol, or a salt, free base, ester, derivative, racemate (+ or − enantiomer) or prodrug thereof) is included in an article of manufacture. In one aspect, an article of manufacture is a container having disposed therein a 5-HT2A receptor agonist or antagonist. In particular aspects, a container comprises a canister having disposed therein contents comprising agonist or antagonist, said contents under pressure. In another aspect, a container comprises an aerosol generator or a spray generator (e.g., an inhaler, nasal sprayer or nebulizer). Exemplary inhalers include metered dose and a dry powder inhalers. In a further aspect, an article of manufacture is for delivery of agonist or antagonist to the lungs or airways, for example, an intubation tube or face mask.

In accordance with the invention, yet further provided are kits. In one embodiment, a kit includes a 5-HT2A receptor agonist or antagonist (e.g., (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer) or prodrug thereof), an article of manufacture for delivery of the antagonist to the lungs or airways, and instructions for administering said 5-HT2A receptor antagonist to the lungs or airways of a subject. In a particular aspect, a kit includes a second drug (e.g., an anti-inflammatory, anti-asthmatic or anti-allergy drug; a hormone or a steroid; an anti-histamine, anti-leukotriene, anti-IgE, anti-α4 integin, anti-β2 integrin, anti-CCR3 antagonist, β2 agonist, anti-selectin or glucocorticoid; H1-receptor antagonist; or a xanthine drug). In more particular aspects, an anti-leukotriene is a cysteinyl-leukotriene (Cys-LT); a β2 agonist is a β2-adrenoceptor.

### BRIEF DESCRIPTION OF DRAWINGS

FIG. 1 illustrates data indicating that 5-HT-induced chemotaxis is selective for eosinophils and does not detectably induce chemotaxis of neutrophils.

FIG. 2 illustrates data indicating that migration induced by 5-HT was a directed chemotactic response and not chemokinetic.

FIG. 3 illustrates data indicating a direct role for 5-HT2A receptor in 5-HT-mediated chemotaxis of human eosinophils, and the ability of MDL-100,907 to block this chemotaxis.

FIG. 4 illustrates data indicating inhibition of airway hyperresponsiveness (AHR) in allergen-challenged 5-HT2A receptor knock out (5-HT2A^{-/-}) mice.

FIG. 5 demonstrates that MDL-100,907 is an effective inhibitor of eosinophil activity and chemotactic response.

FIG. 6 illustrates data indicating that MDL-100,907 inhibits AHR in OVA-challenged mice.

FIG. 7 illustrates data indicating that MDL-100,907 inhibited the number of eosinophils present in the BAL fluid collected from OVA-challenged mice.

### DETAILED DESCRIPTION

In accordance with the invention, there are provided methods of modulating eosinophil migration, chemotaxis or generation. In one embodiment, a method includes contacting eosinophils with an amount of a binding agent (e.g., a 5-HT2A receptor agonist or antagonist) sufficient to modulate eosinophil migration, chemotaxis or generation.

The term “modulate” and grammatical variations thereof include any detectable response, or change in the referenced property or characteristic. The term modulate therefore can mean to reduce, inhibit, decrease, delay, halt, eliminate or prevent, or induce, stimulate, promote, enhance or increase. Thus, when used in reference to eosinophil migration, chemotaxis or generation, the term modulate includes reduce, inhibit, decrease, delay, halt, prevent, induce, stimulate, promote or increase eosinophil migration, chemotaxis or generation.

The term “migration” when used in reference to an eosinophil, means the ability of an eosinophil or eosinophil progenitor cell to move from one location to another in vitro, for example, to move between or within a tissue or organ, bloodstream, or a subject. For example, eosinophils can migrate from bone marrow to lumen of tissue (e.g., airway, lung, intestine). Typically, eosinophils roll along blood vessel walls and under go adhesion and extravasation into the target tissue or organ. Thus, migration refers to any eosinophil or eosinophil progenitor cell movement in vitro locally, regionally, or systemically. Eosiniophil migration can be quantitated using transmigration and other assays known in the art (see, for example, Examples 1 and 2; Yamamato et al., Am. J. Respir. Cell Mol. Biol., 23: 379 (2000); and. Nagase et al., Int. Arch. Allergy and Immunology 122:10 (2000), which describes an assay system based on eosinophil peroxidase (EPO) determination).

The term “chemotaxis” and grammatical variations thereof when used in reference to an eosinophil, means the response of an eosinophil or eosinophil progenitor cell to a chemical signal that results in the eosinophil or eosinophil progenitor cell movement being modulated by the signal. Chemoattractants such as lipopolysaccharide (LPS) and other foreign antigens, as well as chemokines (e.g., eotaxin) and cytokines, can induce or stimulate movement of eosinophils or eosinophil progenitor cells towards the chemical signal. Other chemical signals can induce or stimulate movement of eosiniophils or eosinophil progenitor cells away from the chemical signal. Eosiniophil chemotaxis can be assessed using transmigration and other assays known in the art (see, for example, Examples 1 and 2; and Nagase et al., Int. Arch. Allergy and Immunology 122:10 (2000)).

The term “generation” and grammatical variations thereof when used in reference to an eosinophil, refers to the proliferation, differentiation, survival, production, accumulation of eosinophils or eosinophil progenitor cells. Eosinophil generation can be detected by direct or indirect assays of eosinophil or eosinophil progenitor cell numbers or counts, locally or regionally, in a given area (e.g., tissue or organ), or systemically. For example, bronchiolar lavage fluid (BAL) can be collected from animals in order to ascertain numbers or counts of eosinophils in the fluid (see, for example, Example 1).

In accordance with the invention, there are also provided methods of modulating eosinophil migration, chemotaxis or generation by contact with or administration of a binding agent. In one embodiment, the binding agent includes an agonist or antagonist. In another embodiment, the binding agent includes a 5-HT2A receptor agonist or antagonist. In various aspects, a 5-HT2A receptor antagonist comprises a 4-piperidine-methanol or N-aralkyl-piperidine-methanol derivative, such as (+)-α-(2,3-dimethoxyphenyl)-1-[2-(4-fluorophenyl)ethyl]-4-piperidinemethanol (MDL-100,907) or a salt, free base, ester, derivative, racemate (+ or − enantiomer mixture), or a prodrug thereof. In additional aspects, a 4-piperidine-methanol or N-aralkyl-piperidine-methanol derivative is selected from: α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; and α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer mixture), or prodrug thereof.

Representative non-limiting structures may be represented by the formula (I): 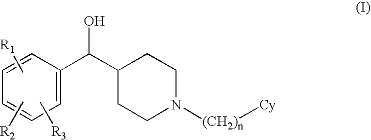
their optical isomers and mixtures thereof, the pharmaceutically acceptable salts thereof. In Formula I, the variables have the following meanings: n is 2, 3 or 4; Cy is selected from the group consisting of optionally substituted aryl, optionally substituted cycloalkyl, optionally substituted heteroaryl, and optionally substituted heterocycloalkyl; examples of aryl include phenyl and napthyl; examples of cycloalkyl include C₅₋₆ cycloalkyls; examples of heteroaryls include pyridinyl, pyrazinyl, pyrimidyl and the like; examples of heterocycloalkyl include piperidinyl, tetrahydrofuryl, tetrahydrpyranyl, and morpholinyl; optional substituents on Cy are selected from the group consisting of halogen, C₁₋₆ alkyl, C₁₋₂ haloalkyl, C₁₋₂ perhaloalkyl, C₁₋₆ alkoxy, C₁₋₆ haloalkoxy, and C₁₋₂ perhaloalkoxy; each R¹, R², and R³ are independently selected from the group consisting of hydrogen, C₁₋₆ alkyl, halogen, haloalkyl, perhaloalkyl, hydroxy, C₁₋₆ alkoxy, C₁₋₆ haloalkoxy, C₁₋₂ perhaloalkoxy or amino; representative R¹, R², and R³ alkyl substituents include but are not limited to methyl, ethyl, propyl, isopropyl, n-butyl, t-butyl, pentyl, hexyl, cyclopropyl, cyclopentyl; the term “halogen” includes fluoro, chloro, bromo, and iodo; representative C₁₋₆ alkoxy substituents include methoxy, ethoxy, isopropoxy and such of the aforementioned alkyl groups attached thru an oxygen. In those instances wherein R¹, R² or R³ are other than hydrogen, the substituents may be located at any available position, ortho, meta or para to the existing hydroxylated methylene for monosubstituted phenyl moieties. The 2,3-, 2,4-, 2,5-, 3,4-, or 3,5-disubstituted phenyl moieties and 3,4,5-, 2,3,5-, 2,3,4-trisubstituted phenyl moieties are embraced herein. The compounds of Formula I contain an asymmetric carbon atom and thus exist in optically isomeric forms. Individual optical isomers and the mixtures thereof are within the scope of the invention. Such mixtures or pure optical isomers can be separated by methods known in the art. Specific embodiments of the invention include the following:

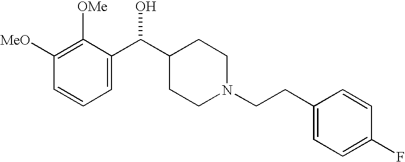

### R-(+)-α-(2,3-dimethoxyphenyl)-1-[2-(4-fluorophenyl)ethyl]-4-piperidine methanol

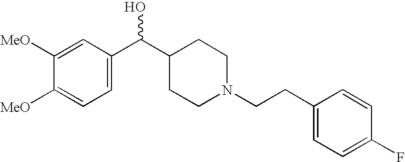

### α-(3,4-dimethoxyphenyl)-1-[2-(4-fluorophenyl)ethyl]-4-piperidine methanol

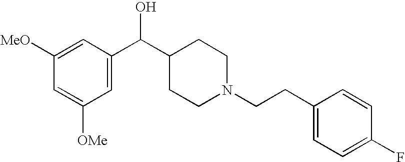

### α-(3,5-dimethoxyphenyl)-1-[2-(4-fluorophenyl)ethyl]-4-piperidine methanol

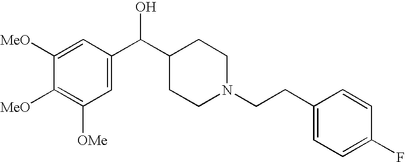

### α-(3,4,5-trimethoxyphenyl)-1-[2-(4-fluorophenyl)ethyl]-4-piperidine methanol

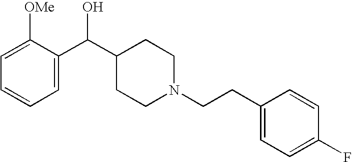

### α-(2-methoxyphenyl)-1-[2-(4-fluorophenyl)ethyl]-4-piperidine methanol

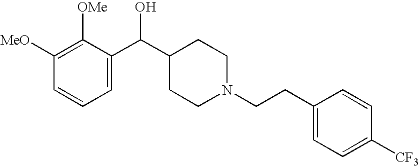

### α-(2,3-dimethoxyphenyl)-1-[2-(4-trifluormethylphenyl)ethyl]-4-piperidine methanol

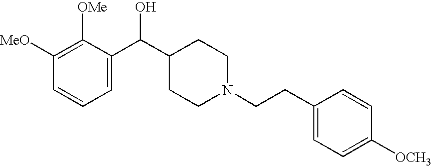

### α-(2,3-dimethoxyphenyl)-1-[2-(4-methoxyphenyl)ethyl]-4-piperidine methanol

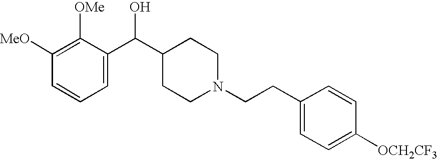

### α-(2,3-dimethoxyphenyl)-1-[2-(4-(2′,2′,2′-trifluoroethoxyphenyl)ethyl]-4-piperidine methanol

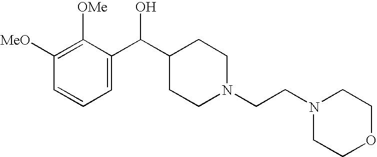

### α-(2,3-dimethoxyphenyl)-1-[2-{(morpholino)ethyl}]-4-piperidine methanol

Embodiments of the invention possess one or more chiral centers and each center may exist in the R or S configuration, giving rise to numerous enantiomeric and diastereomeric forms of the same molecular formula. The invention includes all diastereomeric, enantiomeric, and epimeric forms as well as the appropriate mixtures thereof.

The term “binding agent,” or “binding compound,” when used in reference to a molecule, means that the molecule can directly or indirectly bind to another molecule, at least transiently. Binding agents or binding compounds include agents and compounds that bind to or interact with 5-HT2A receptor, directly or indirectly.

Binding agents and compounds include agents and compounds that can modulate an activity or expression of the molecule to which the agent or compound binds. Binding agents therefore include agents that can increase, stimulate, induce, enhance or promote an activity or expression of the molecule to which the agent or compound binds. Binding agents include agents that can decrease, reduce, inhibit, delay, halt, eliminate or prevent an activity or expression of the molecule to which the agent or compound binds. Binding agents or compounds that bind to or interact with 5-HT2A receptor and increase, stimulate, induce, enhance or promote an activity or expression of 5-HT2A receptor can be referred to as. 5-HT2A receptor agonists. Binding agents or compounds that decrease, reduce, inhibit, delay, halt, eliminate or prevent an activity or expression of 5-HT2A receptor can be referred to as 5-HT2A receptor antagonists.

5-HT2A receptor agonists can be identified using assays known in the art. For example, 5-HT2A receptor agonists elicit a characteristic head twitch response in vivo (e.g., in mice). In brief, administration of a 5-HT2A receptor agonist such as serotonin induces a head twitch in mice. Thus, 5-HT2A receptor agonists can be identified as such using the head twitch assay.

5-HT2A receptor antagonists have an ability to antagonize the 5HT2 receptor and can therefore be identified by inhibiting or blocking the effect of serotonin, as determined using the head twitch assay (Friedman et al., Commun. Psychopharmacol. 3:89 (1979)). In brief, administration of 5-methoxy-N,N-dimethyltryptamine (5-MeO-DMT) to mice produces a characteristic head twitch in the mice. To test for antagonist activity, mice are administered 5-MeO-DMT and a test antagonist. An absence of head twitches in the mice indicates the ability of the test antagonist to antagonize the 5HT₂ receptor in vivo. The potency in which the antagonist inhibits head twitches correlates with the affinity of the antagonist for 5-HT2 receptors.

Particular examples of 5-HT2A receptor antagonists include 4-piperidine-methanol or N-aralkyl-piperidine-methanol derivatives, such as (+)-α-(2,3-dimethoxyphenyl)-1-[2-(4-fluorophenyl)ethyl]-4-piperidinemethanol (MDL-100,907) or a salt, free base, ester, derivative, racemate (+ or − enantiomer mixture), or a prodrug thereof. In additional aspects, a 4-piperidine-methanol or N-aralkyl-piperidine-methanol derivative is selected from: α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; and α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer mixture), or prodrug thereof. Additional examples of 5-HT2A receptor antagonists include cyproheptidine (CYP), methysergide, ketanserin, pirenperone, ritanserin, clozapine, MDL 28,133A and methiothepin.

Binding agents can bind selectively or non-selectively. Selective or non-selective binding may occur in solution, in solid phase, in vitro, ex vivo or in vivo. 5-HT2A receptor binding agents therefore include agents that selectively or non-selectively, directly or indirectly, bind to or interact with 5-HT2A receptor. Agents that bind to and modulate 5-HT2A receptor activity or expression selectively or non-selectively are referred to as selective or non-selective 5-HT2A receptor agonists or antagonists.

As used herein, the term “selective” when used in reference to a binding agent such as a 5-HT2A receptor agonist or antagonist, means that the agonist or antagonist binds with specificity to the target entity (e.g., 5-HT2A receptor) and does not significantly cross-react with other non-target entities. For 5-HT2A receptor, selectivity means that the binding agent does not bind to and stimulate significant, or induces little if any detectable agonist or antagonist activity of 5HT1, adrenergic alpha-1, dopamine D-2 or muscarinic cholinergic receptors. 5-HT2A receptor selective binding agents are such that binding to or interaction with one or more of 5HT1, adrenergic alpha-1, dopamine D-2 or muscarinic cholinergic receptor may occur, but will not stimulate or induce significant agonist or antagonist activity of one or more of such non-5-HT2A receptors to the extent that the activity causes a physiological effect that eliminates the therapeutic effect of the selective binding agent, or substantially reduces or decreases, the therapeutic value due to the type, frequency, severity, probability or susceptibility of one or more adverse or undesirable side effects.

A non-selective binding agent means that the agonist or antagonist is not selective for the entity to which it binds, i.e., it cross-reacts with other entities. For 5-HT2A receptor, non-selective agonists or antagonists may therefore bind to or interact with 5-HT2A receptor as well as other substances. For example, a non-selective 5-HT2A receptor binding agent may modulate activity or expression of one or more 5HT1, adrenergic alpha-1, dopamine D-2 or muscarinic cholinergic receptors. Such non-selective 5-HT2A receptor binding agents may be used in a method of the invention. A non-selective 5-HT2A receptor binding agent that stimulates or induces agonist or antagonist activity of one or more of 5HT1, adrenergic alpha-1, dopamine D-2 or muscarinic cholinergic receptors to the extent that the activity causes a physiological effect that eliminates or substantially reduces or decreases the therapeutic value of the non-selective 5-HT2A receptor non-selective 5-HT2A receptor binding agent need not be employed and can be optionally excluded.

Non-limiting examples of non-selective 5-HT2A receptor binding agents include cyproheptidine (CYP), methysergide, ketanserin and pirenperone. These and other agents bind to other receptors, such as 5HT1, adrenergic alpha-1, dopamine D-2 or muscarinic cholinergic receptors. Binding agents that function to antagonize a 5-HT2A receptor activity identified in the head twitch assay may therefore also bind to and potentially modulate activity or expression of other receptors (e.g., 5HT1, adrenergic alpha-1, dopamine D-2 or muscarinic cholinergic receptors).

Selective 5-HT2A receptor agonists or antagonists can be identified by assays known in the art. For example, anesthetized ganglion-blocked dog can be used to detect alpha-adrenergic and sertoninergic inhibition or blockade. A binding agent that antagonizes serotonin in vivo (e.g., eliminates the pressor response to serotonin) but has no apparent effect on phenylephrine in vivo indicates in vivo antagonism of serotonin but little, if any, alpha-adrenergic receptor antagonist activity. Ketanserin, which blocks both serotonin and phenylephrine in vivo, can be used as a positive control.

Non-limiting examples of selective 5-HT2A receptor binding agents include, for example, (+)-α-(2,3-dimethoxyphenyl)-1-[2-(4-fluorophenyl)ethyl]-4-piperidinemethanol (MDL-100,907) and salts, free bases, esters and prodrugs thereof. The 5-HT2A receptor antagonist (+)-α-(2,3-dimethoxyphenyl)-1-[2-(4-fluorophenyl)ethyl]-4-piperidinemethanol (MDL-100,907) exhibits little detectable activity at the 5HT1, adrenergic alpha-1, dopamine D-2 or muscarinic cholinergic receptors. A 4-piperidine-methanol or N-aralkyl-piperidine-methanol derivative will also likely be selective for 5-HT2A receptor, as will (+)-α-(2,3-dimethoxyphenyl)-1-[2-(4-fluorophenyl)ethyl]-4-piperidinemethanol (MDL-100,907) derivatives, racemate (+ or − enantiomer mixture), and prodrugs thereof.

Binding agents also include variants and derivatives that retain at least a part or all of an activity of the non-variant or non-derivatized binding agent. Non-limiting examples of activities that can be retained, at least in part, include agonist or antagonist activity, affinity (e.g., K_{d}), avidity and binding selectivity (specificity) or non-selectivity. The variant or derivatized binding agent can exhibit an activity that is greater or less than a corresponding non-variant or non-derivatized binding agent, e.g., greater or less agonist or antagonist activity, affinity (e.g., K_{d}), avidity or binding selectivity (specificity) or non-selectivity. For example, “at least a part” of an activity of a binding agent can be when the variant or derivatized agent has less of an agonist or antagonist activity, e.g., 10-25%, 25-50%, 50-60%, 60-70%, 70-75%, 75-80%, 80-85%, 85-90%, 90-95%, 95-99%, 100%, or any percent or numerical value or range or value within such ranges. An activity of a binding agent can be when the variant or derivatized agent has more of an agonist or antagonist activity, e.g., 110-125%, 125-150%, 150-175%, 175-200%, 200-250%, 250-300%, 300-400%, 400-500%, 500-1000%, 1000-2000%, 2000-5000%, or more, or any percent or numerical value or range or value within such ranges. At least a part of binding affinity of a binding agent can be when the variant or derivatized agent has less affinity, e.g., 1-3-fold, 1-5-fold, 2-5 fold, 5-10-fold, 5-15-fold, 10-15-fold, 15-20-fold, 20-25-fold, 25-30-fold, 30-50-fold, 50-100 fold, 100-500-fold 500-1000-fold, 1000-5000-fold, or less (e.g., K_{d}), or any numerical value or range of values within such ranges. At least a part of binding affinity of a binding agent can be when the variant or derivatized agent has more affinity, e.g., 1-3-fold, 1-5-fold, 2-5 fold, 5-10-fold, 5-15-fold, 10-15-fold, 15-20-fold, 20-25-fold, 25-30-fold, 30-50-fold, 50-100 fold, 100-500-fold 500-1000-fold, 1000-5000-fold, or more (e.g., K_{d}), or any numerical value or range of values within such ranges.

The amount of activity can be assessed directly, such as measuring the particular activity (e.g., agonist or antagonist activity, binding affinity, avidity, selectivity (specificity) or non-selectivity. For example, 5-HT2A receptor agonist activity can be measured using the head twitch assay. 5-HT2A receptor antagonist activity antagonist activity can be measured by the ability of the antagonist to inhibit or block the head twitch. Binding affinity, avidity or selectivity (specificity) can be detected or measured using competition assays with other binging agents.

Activity of a binding agent can also be assessed indirectly using an appropriate assay, such as an assay that detects and quantifies agonist or antagonist activity. Agonist or antagonist activity of a given binding agent is likely to be proportional with the binding affinity or binding selectivity of the agent and, therefore, can be measured indirectly by determining binding affinity or selectivity (specificity) of the agent. For example, activity of a 5-HT2A receptor agonist is typically proportional to binding affinity; binding affinity is a measure of the energy of binding between the binding combination. Thus, binding affinity can be an indirect measure of 5-HT2A receptor agonist activity. Binding affinity can be represented quantitatively by association (Kₐ) or dissociation (K_{d}) rate. Equilibrium affinity constant, K, is the ratio of Kₐ/K_{d}. Typically, binding affinity is quantitatively expressed as dissociation (K_{d}) rate.

A retained activity, such as variant or derivative of a 5-HT2A receptor agonist or antagonist, can be the same or substantially the same as the comparison or reference binding agent. As used herein, the term “the same,” when used in reference to agonist or antagonist activity means that the activity is within about 50% more than or less than the agonist or antagonist activity. The term “substantially the same” when used in reference to agonist or antagonist activity means that the activity is within about 100-500% (2-5-fold) or any percent value or range of percent values within such ranges, more than or less than the agonist or antagonist activity. The same, when used in reference to binding affinity, means that the dissociation constant (K_{d}) is within about 1 to 5-fold, or any numerical value or range of values within such a range, of the referenced agent (e.g., 1-5 fold greater affinity or 1-5 fold less affinity than the reference agent). The term “substantially the same” when used in reference to binding affinity, means that the dissociation constant (K_{d}) is within about 5 to 100 fold, or any numerical value or range of values within such a range, of the reference agent (5-100 fold greater affinity or 5-100 fold less affinity than the reference agent). The term “the same,” when used in reference to association constant (Kₐ) is within about 1 to 5 fold, or any numerical value or range of values within such a range, of the reference agent (within 1-5 fold greater or 1-5 fold less than the association constant, Kₐ, of the reference agent). The term “substantially the same” when used in reference to association constant (Kₐ), means that the association constant is within about 5 to 100 fold greater or less, or any numerical value or range of values within such a range, than the association constant, Kₐ, of the reference agent (5-100 fold greater or 5-100 fold less than the reference agent).

Dissociation (K_{d}) constants can be measured using radolabeled binding agent in competitive binding assays with increasing amounts of unlabelled binding agent to generate saturation curves. The receptor used in the binding assay can be expressed in vitro, on cells or be present in extracts. Association (Kₐ) and dissociation (K_{d}) constants can be measured using surface plasmon resonance (SPR) (Rich and Myszka, Curr. Opin. Biotechnol. 11:54 (2000); Englebienne, Analyst. 123:1599 (1998)). SPR methods for real time detection and monitoring of protein binding rates are known and are commercially available and can be used to determine dissociation (K_{d}) constants (BiaCore 2000, Biacore AB, Upsala, Sweden; and Malmqvist, Biochem. Soc. Trans. 27:335 (1999)).

As discussed, a particular activity of a variant or derivative of a binding agent may be less than or greater than the activity of a corresponding non-variant or non-derivatized binding agent. For example, a 5-HT2A receptor agonist variant or derivative may have less or greater agonist activity than non-vanant or non-derivatized 5-HT2A receptor agonist; and a 5-HT2A receptor antagonist variant or derivative may have less or greater antagonist activity than non-vanant or non-derivatized 5-HT2A receptor antagonist.

Specific non-limiting examples of variants and derivatives include salts, free-bases, esters, racemates (+ or − enantiomer), prodrugs and mixtures thereof. Methods of producing or isolating salts, free-bases, esters (+ or − enantiomer mixture)s, prodrugs and mixtures thereof are disclosed herein and known in the art (see, for example, for example, J. March “Advanced Organic Synthesis,” 3rd Ed; Wiley 1985; Smith, M. B. and March, J. “March's Advanced Organic Chemistry: Reactions, Mechanisms, and Structure,” 5th Ed; Wiley 2000).

Additional non-limiting examples of variants and derivatives include minor changes or alterations of a chemical group, linkage or covalent bond within a particular binding agent. For example, modifications of 4-piperidine-methanol and N-aralkyl-piperidine-methanol derivatives, including modifications of formula (I), as set forth herein.

Preparation of the compounds useful in the invention may be achieved by standard chemical reactions known in the art, with reference to the synthesis scheme below.

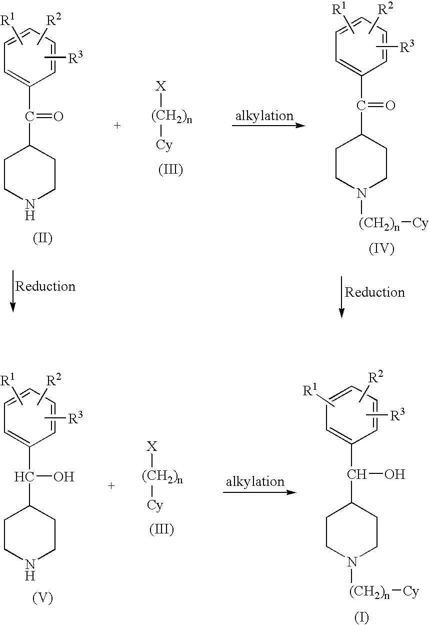

Moving clockwise, beginning at the upper left, N-alkylation with (III) of an optionally substituted phenyl 4-piperidinemethanone (II) produces an N-alkylated phenyl-4-piperidinyl methanone intermediate (IV), which intermediate is then chemically reduced to the product (I). Alternately, and moving counterclockwise beginning at the upper left, the ketone moiety of an optionally substituted phenyl 4-piperidinemethanone (II) may be chemically reduced to an alcohol intermediate (V) and that intermediate may be N-alkylated with (III) to the desired product (I). The chemical reduction of a ketone to its corresponding alcohols may be effected by standard reduction procedures such as by catalytic hydrogenation or metal hydride reduction using sodium or potassium borohydride. In either case, in those instances wherein an R-substituent represents a reactive hydroxy group, then such group should first be protected (using such standard protecting groups as acetate, trifluoroacetate, benzyloxy, benzyloxycarbonyl, methoxymethyl, ethoxymethyl, and the like) and then, following the foregoing N-alkylation-reduction reactions, the protecting group is removed. Standard techniques for protecting and de-protecting hydroxy functions are well known in the art. Analogously in those instances wherein any R group represents an amino moiety, it is preferred to prepare the appropriate nitro analog and, as a last step, reduce the nitro group to the desired amino moiety.

Suitable N-alkylating reagents, X—(CH₂)ₙ-Cy, wherein X is a suitable leaving group (e.g., halide, tosylate or other functional equivalent thereof) and n=2, Cy is 4-morpholino or optionally substituted phenyl include but are not limited to the following:

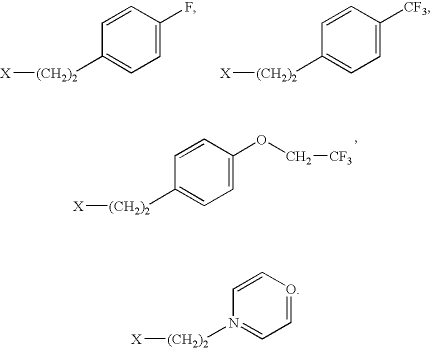

The morpholino group may have increased solubility and metabolic properties compared to the 4-fluoro analog.

N-alkylation procedures are effected by reacting about equimolar quantities of the N-alkylating reactants (III) with an appropriately substituted 4-piperidinemethanol (II) or 4-piperidinylmethanone (V), in a suitable solvent, in the presence of an acid acceptor (e.g., the carbonates or bicarbonates of potassium or sodium, or an excess of the piperidine reactant), optionally with a small amount of potassium iodide being present. Suitable solvents are toluene, xylene, chlorobenzene, N,N-dimethylformamide, N,N-dimethylacetamide, ketones (acetone, butanone, MIBK, cyclohexanone, cyclopentanone, etc.) and alcohols (ethanol, propanol, butanol, etc.) The reaction mixture is heated over a wide range of temperatures (50° C.-180° C.) depending on the reflux temperatures of the reaction mixture. The reaction is continued until completed, generally a period of hours or days.

After completion of the reaction, the reaction mixture is filtered, the product optionally converted to its mineral or organic acid salt and the product is recrystallized by techniques known in the art. Suitable solvents for recrystallization are methanol, ethanol, isopropanol, butanone, acetone, ethyl acetate, and diethylether.

Optionally substituted phenyl 4-piperidyl ketone intermediates (II) used in the preparation of the invention may be prepared by a Friedel-Craft reaction of benzene or a optionally substituted benzene with isonipecotic acid chloride HCl or N-(trifluoroacetyl)isonipecotic trifluoroacetic anhydride followed by aqueous potassium carbonate hydrolysis in the latter case. Reaction of an optionally substituted phenyl Grignard reagent with 4-cyanopiperidine (prepared by hydrolyzing N-trifluoroacetyl-4-cyanopiperidine) will also yield the intermediate ketones. The method enables the synthesis of several substituted phenyl derivatives without isomeric impurities.

Alternative methods of synthesis of the compounds of this invention include the reactions of either an optionally substituted phenylmagnesium halide or a optionally substituted phenyl lithium reactant with 1-(substituted-phenyl)-4-piperidine carboxaldehyde. Another alternative procedure includes the catalytic hydrogenation of an alpha-(R-substituted phenylmethanol)-1-(R,R¹-phenylalkyl)pyridinium halide, or its ketone analog, to the desired product. Still another method is the chemical reduction of a substituted-phenyl[-1-(2-R,R¹-phenacyl]-4-piperidinyl)-methanone to the desired products of this invention. For these foregoing reactions standard procedures well known in the art may be used in the preparation of the necessary intermediates as well as for the final step producing the desired products.

A synthesis of one species useful in the invention beginning with commercially available 2,3-dimethoxybenzaldehyde (available from Aldrich Chemical Co.) is shown in the following scheme:

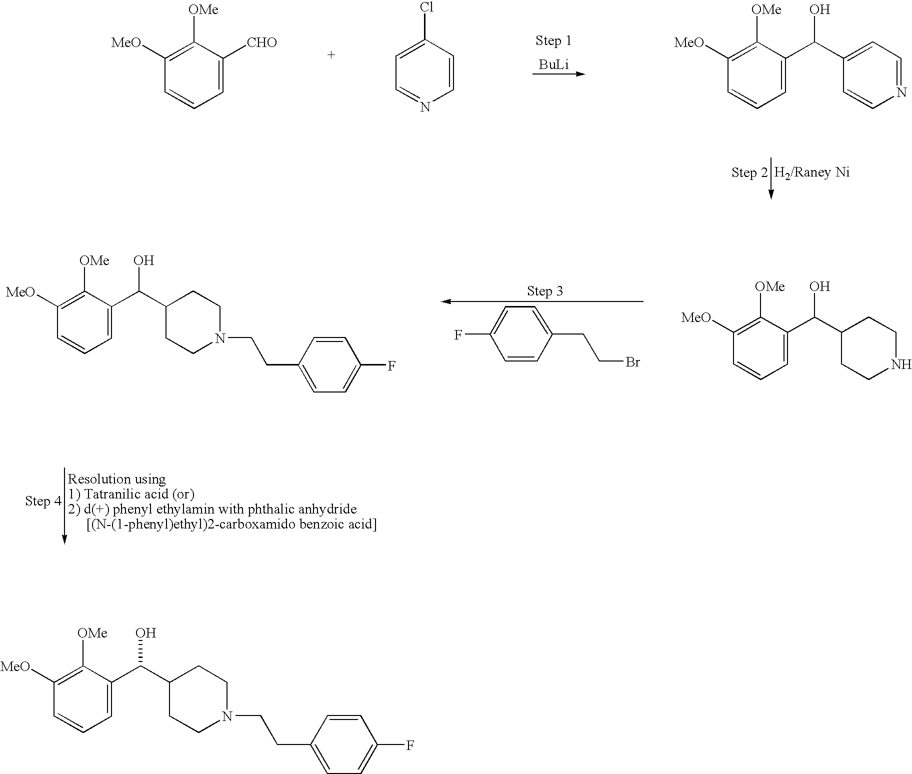

The method outlined in the scheme above features a carbon-carbon bond formation (Step 1) with a resulting formation of a carbinol moiety. This method is general and is suitable for preparing numerous embodiments when starting with the appropriate aldehyde precursor. Step 2 features a transition metal-catalyzed reduction of the pyridyl moiety to a piperidine moiety. Hydrogenation with Pd/C or the less expensive and recyclable use of Raney Ni will affect this transformation. Step 3 features the attachment of the piperidinyl nitrogen to an organic electrophile. This step is versatile and allows the attachment of a variety of short-chain carbon electrophiles bearing Cy moieties as described above. The resolving agent in final Step 4 is stable and readily available. The method of synthesis is exemplified by the following non-limiting example of one embodiment.

### Synthesis of R-(+)-α-(2,3-dimethoxyphenyl)-1-[2-(4-fluorophenyl)ethyl]-4-piperidine methanol

Step 1: Synthesis of α-(2,3-dimethoxyphenyl)-4-pyridyl methanol

To butyl lithium (50 ml, 2.5M solution in hexane, 120 mmol) was added 4-chloropyridine (12.5 g, 110 mmol) in THF (50 ml) at −70° C. The mixture was stirred for 30 minutes at that temperature and then 2,3-dimethoxy benzaldehyde (16.6 g, 100 mmol) was added and the stirring continued for half-an-hour while the temperature slowly rose to room temperature. The reaction was quenched with water and the resulting mixture extracted with dichloromethane (2×50 ml). Evaporation of the solvent gave the desired product.

### Step 2: Synthesis of α-(2,3-dimethoxyphenyl)-4-piperidine methanol

The product from Step 1 (14 g, 57 mmol) was dissolved in ethanol (70 ml) and then Raney-Ni was added (2.8 g). Hydrogenation was carried out at 60° C. at a pressure of 20 atmospheres. After reaction was complete, the catalyst was filtered and the solvent evaporated to get the product.

### Step 3: Synthesis of α-(2,3-dimethoxyphenyl)-1-[2-(4-fluorophenyl)ethyl]4-piperidine methanol

The product from step 2 (10 g, 40 mmol) was dissolved in DMF(100 ml) and 4-fluorophenylethyl bromide (8.9 g, 44 mmol) and potassium carbonate (10 g, 72 mmol) were added. The reaction was heated to 100° C. and stirred at that temperature for 4 hours. Filtration of inorganics and evaporation of DMF to gave the product.

### Step 4: Resolution of R-(+)-α-(2,3-methoxyphenyl)-1-[2-(4-fluorophenyl)ethyl]-4-piperidine methanol

The racemic mixture from step-3 was resolved using (N-(1-phenyl)ethyl)-2-carboxamido benzoic acid (prepared from (+) phenyl ethylamine and phthalic anhydride) in various solvents using standard crystallization procedures.

As used herein, the term “contact” and grammatical variations thereof means a physical or functional interaction between one entity and one or more other entities. An example of physical contact is a direct or indirect binding, such as between a binding agent and a receptor (e.g., a 5-HT2A receptor agonist or antagonist and 5-HT2A receptor). An example of a functional interaction is where an intermediate facilitates or mediates a change in activity of one entity by another entity, such as a signaling pathway where molecules within the pathway functionally interact but need not physically contact each other. In the methods, contact can occur in solution, in solid phase, in vitro, ex vivo or in vivo (i.e., in a subject).

In accordance with the invention, there are provided methods of modulating eosinophil migration, chemotaxis or generation in solution, in solid phase, in vitro, ex vivo or in vivo (i.e., in a subject). In one embodiment, a method includes contacting or administering to a subject, e.g. a subject in need thereof, an amount of a 5-HT2A receptor agonist or antagonist to treat the subject, e.g. a subject in need thereof. In various particular aspects, an amount of 5-HT2A receptor agonist contacted with or administered to the subject is sufficient to increase, stimulate, promote or induce eosinophil migration, chemotaxis or generation in a tissue or organ. In additional particular aspects, an amount of 5-HT2A receptor antagonist contacted with or administered to the subject is sufficient to reduce, decrease, inhibit, delay, halt, eliminate or prevent eosinophil migration, chemotaxis or generation in a pulmonary tissue or organ, gut or bone marrow; or is sufficient to reduce, decrease, inhibit, delay, halt, eliminate or prevent eosinophil migration, chemotaxis or generation in lung, airways or respiratory mucosa. In another aspect, an amount of 5-HT2A receptor antagonist is contacted with or administered to a subject whom has previously experienced an asthmatic episode or airway-constriction or obstruction, or is in need of airway-dilation.

As used herein, the term “subject” includes animals, typically mammalian animals, such as but not limited to humans, non-human primates (apes, gibbons, chimpanzees, orangutans, macaques), domestic animals (dogs and cats), farm animals (horses, cows, goats, sheep, pigs), and experimental animals (mouse, rat, rabbit, guinea pig). Subjects include animal disease models (e.g., asthma, allergy). Subjects include naturally occurring or non-naturally occurring mutated or non-human genetically engineered (e.g., transgenic or knockout) animals. Subjects further include animals having or at risk of having a chronic or acute condition, disorder or disease.

As used herein, the term “associated with,” when used in reference to the relationship between a symptom and a condition, disorder or disease, means that the symptom is caused by the condition, disorder or disease, or is a secondary effect of the condition, disorder or disease. A symptom that is present in a subject may therefore be the direct result of or caused by the condition, or may be due at least in part to the subject reacting or responding to the condition, disorder or disease. For example, symptoms that occur during an allergic episode are due in part to hypersensitivity or an aberrant response of the immune system of the subject to the allergen.

In accordance with the invention, there are provided methods of treating a subject having or at risk of having a condition associated with undesirable or abnormal eosinophil migration, chemotaxis or generation. In one embodiment, a method includes administering to a subject an amount of 5-HT2A receptor antagonist sufficient to reduce undesirable or abnormal eosinophil migration, chemotaxis or generation to treat the subject. In various aspects, a method reduces, inhibits, decreases, eliminates, delays, halts, or prevents one or more symptoms (adverse or undesirable) associated with or caused by the condition, disorder or disease.

Subjects in need of treatment in accordance with the invention include subjects having or at risk of having undesirable or abnormal eosinophil migration, chemotaxis or generation, or a condition, disorder or disease associated with eosinophil migration, chemotaxis or generation. A subject is considered to be in need of treatment where a method is considered appropriate for treating a condition, disorder or disease associated with eosinophil migration, chemotaxis or generation, as such treatment can provide a beneficial effect, such as reducing, inhibiting, decreasing, delaying, halting, eliminating or preventing progression, severity, frequency, duration, susceptibility or probability of developing one or more symptoms associated with the condition, disorder or disease.

Conditions associated with undesirable or abnormal eosinophil migration, chemotaxis or generation include, for example, chronic or acute inflammatory conditions, disorders and diseases, allergies, allergic conditions, disorders and diseases. An “inflammatory” condition, disorder or disease refers to one or more physiological responses that characterize or constitute inflammation. An “allergy” or “allergic condition,” as used herein refers to a hypersensitivity to a substance (e.g., an allergen). Allergic conditions, disorders and diseases include but are not limited to allergic asthma, hayfever (seasonal rhinitis), allergic rhinitis, allergic conjunctivitis, eczema, urticaria, food allergies, and other atopic conditions.

Inflammatory, allergic and non-allergic and conditions, disorders and diseases of the respiratory system, including airways and lung, include asthma, chronic obstructive pulmonary disease (“COPD”), granulomatus diseases of the lungs and lower airway passages, non-malignant proliferative disease of the lungs e.g., idiopathic pulmonary fibrosis, hypersensitivity pneumonitis and bronchopulmonary dysplasia. Non-limiting examples of allergic conditions, disorders and diseases include, for example, extrinsic bronchial asthma; allergic rhinitis (AR); Onchocercal dermatitis; atopic dermatitis, drug reactions; nodules, eosinophilia, rheumatism, dermatitis, and swelling (NERDS); esophageal and gastro-intestinal (GI) allergies.

In accordance with the invention, there are provided methods of reducing progression, severity, frequency, duration, susceptibility or probability of inflammatory, allergic and non-allergic conditions, disorders and diseases of the respiratory system. In one embodiment, a method includes administering to a subject an amount of 5-HT2A receptor antagonist sufficient to reduce or decrease progression, severity, frequency, duration, susceptibility or probability of one or more adverse symptoms associated with inflammation in an organ or tissue. In another embodiment, a method includes administering to a subject an amount of 5-HT2A receptor antagonist sufficient to reduce or decrease progression, severity, frequency, duration, susceptibility or probability of one or more adverse symptoms associated with asthma (allergic or non-allergic). In one aspect, the adverse symptom is selected from lung, airway or respiratory mucosum inflammation or tissue damage, shortness of breath, wheezing, coughing, chest-tightness, chest pain, increased heart rate, runny nose, airway-constriction, decreased lung capacity, and an acute asthmatic episode. In another aspect, asthma is associated with exposure to an allergen or associated with exercise. In yet another aspect, the subject has been diagnosed as having asthma.

“Asthma” refers to an allergic or non-allergic condition, disorder or disease of the respiratory system that is episodic and characterized by inflammation with constriction, narrowing or obstruction of the airways. Allergic asthma is typically associated with increased reactivity of respiratory system (airways, lung, etc.) to an inhaled agent. Asthma is frequently, although not exclusively associated with atopic or allergic symptoms. Typically, a subject with asthma suffers from recurrent attacks of paroxysmal dyspnea (i.e., “reversible obstructive airway passage disease”), cough, shortness of breath with wheezing due to spasmodic contraction of the bronchi, sometimes referred to as “bronchospasm,” chest pain, chest tightness, etc. While a plurality of such adverse symptoms typically occur in asthma, the existence of any one is usually adequate for diagnosis of asthma, and for treatment in accordance with the invention.

Asthmatic conditions include allergic asthma as well as bronchial allergy, which typically are provoked by a variety of factors including exercise such as vigorous exercise (“exercise-induced bronchospasm”), and irritant particles (allergens such as pollen, dust, venoms, cotton, dander, foods). Asthmatic conditions can be acute, chronic, mild, moderate or severe asthma.(unstable asthma), nocturnal asthma or asthma associated with psychologic stress.

“Allergic rhinitis” is an allergic reaction of the nasal mucosa (upper airways), which includes hay fever (seasonal allergic rhinitis) and perennial rhinitis (non-seasonal allergic rhinitis) which are typically characterized by seasonal or perennial sneezing, rhinorrhea, nasal congestion, pruritis and eye itching, redness and tearing. “Non-allergic rhinitis” refers to eosinophilic non-allergic rhinitis, in subjects with negative skin tests, and subjects who have abnormal or undesirable numbers of eosinophils in their nasal secretions.

An “allergen” is a substance that can promote, stimulate or induce an allergic or asthmatic episode in a subject. Allergens include plant/tree pollens, insect venoms, animal dander, house dust mite, dust, fungal spores, latex, food and drugs (e.g., penicillin). Examples of particular allergens include proteins specific to the following genera: Canis (Canis familiaris); Dermatophagoides (e.g., Dermatophagoides farinae); Felis (Felis domesticus); Ambrosia (Ambrosia artemiisfolia); Lolium (e.g., Lolium perenne or Lolium multiflorum); Cryptomeria (Cryptomeria japonica); Alternaria (Alternaria alternata); Alder; Alnus (Alnus gultinosa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis or Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis or Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis). Allergens also include peptides and polypeptides used in experimental animal models of allergy and asthma, including ovalbumin (OVA) and Schistosoma mansoni egg antigen.

Conditions associated with undesirable or abnormal eosinophil migration, chemotaxis or generation also include, for example, a vasculitic granulomatous disease. Non-limiting examples of vasculitic granulomatous disease include Temporal vasculitis; Churg-Strauss syndrome; Polyarteritis; Wegener's granulomatosis; Eosinophilic granulomatous prostatitis; and Ulercerative colitis.

Conditions associated with undesirable or abnormal eosinophil migration, chemotaxis or generation further include, for example, an interstitial disorder, pulmonary disorder, and respiratory/respiratory mucosum disorders. Non-limiting examples of interstitial and pulmonary disorders include Eosinophilic pleural effusions; Transient pulmonary eosinophilic infiltrates (Löffler); Histiocytosis; Chronic eosinophilic pneumonia; Hypersensitivity pneumonitis; Allergic bronchopulmonary aspergillosis; Sarcoidosis; Idiopathic pulmonary fibrosis; pulmonary edema; pulmonary embolism; pulmonary emphysema; Pulmonary Hyperventilation; Pulmonary Alveolar Proteinosis; Chronic Obstructive Pulmonary Disease; Interstitial Lung Diseases; and Topical eosinophilia.

A “respiratory disorder” or a “respiratory mucosum disorder” means a condition, disorder or disease related to a tissue or organ of the respiratory system. Examples include, but are not limited to, upper or lower airway inflammations allergy(ies), breathing difficulty, cystic fibrosis (CF), allergic rhinitis (AR), Acute Respiratory Distress Syndrome (ARDS), pulmonary hypertension, lung inflammation, bronchitis, airway obstruction, airway: constriction, airway narrowing, broncho-constriction and inflammation associated with microbial or viral infections, such as picornaviridae (rhinoviruses such as human rhinovirus (HRV); enteroviruses (EV) such as polioviruses, coxsackieviruses and echoviruses; and hepatitis A virus) or severe acute respiratory syndrome (SARS). Additional non-limiting examples of respiratory disorders and respiratory mucosum disorders include apnea, asbestosis, atelectasis; berylliosis, bronchiectasis, bronchiolitis, bronchiolitis obliterans Organizing Pneumonia, Bronchitis, Bronchopulmonary Dysplasia, Common Cold, Cough, Empyema; Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Respiratory Tract Infections, Rhinoscleroma, Scimitar Syndrome, Severe Acute Respiratory Syndrome, Silicosis, Tracheal Stenosis and Whooping Cough.

The term “airway,” as used herein, means a part of or the whole respiratory system of a subject that is exposed to air. “Airways” therefore include the upper and lower airway passages, within which are not limited to the trachea, bronchi, bronchioles, terminal and respiratory bronchioles, alveolar ducts and alveolar sacs. Airways include sinuses, nasal passages, nasal mucosum and nasal epithelium. The airway also includes, but is not limited to throat, larynx, tracheobronchial tree and tonsils.

Conditions associated with undesirable or abnormal eosinophil migration, chemotaxis or generation additionally include, for example, an immunological disorder. Non-limiting examples of immunological disorders include autoimmune diseases, such as, multiple sclerosis; graft rejection and Intrinsic bronchial asthma.

Conditions associated with undesirable or abnormal eosinophil migration, chemotaxis or generation moreover include, for example, hyperproliferative disorder, such as a neoplastic or myeloproliferative disease. Non-limiting examples of neoplastic and myeloproliferative diseases include Hypereosinophilic syndrome.

Subjects having or at risk of having a condition, disorder or disease associated with undesirable or abnormal eosinophil migration, chemotaxis or generation include subjects with an existing condition or a known or a suspected predisposition towards developing a symptom associated with undesirable or abnormal eosinophil migration, chemotaxis or generation, such as the conditions, disorders and diseases set forth herein and know in the art. Thus, the subject can have an active acute or chronic condition, disorder or disease associated with undesirable or abnormal eosinophil migration, chemotaxis or generation, or a latent condition, disorder or disease. At risk subjects include those at risk or predisposed towards suffering from such conditions, disorders or diseases based upon their prior or a family history, but the condition, disorder or disease may not presently manifest itself in the subject.

Particular non-limiting examples of subjects include subjects having or at risk of having an inflammatory or allergic condition, disorder or disease. Non-limiting examples of subjects further include subjects having or at risk of having an adverse or undesirable symptom associated with an inflammatory or allergic condition, disorder or disease. Such at risk subjects can be identified by a personal or family history, through genetic screening, tests appropriate for detection of increased risk, or exhibiting relevant symptoms indicating predisposition or susceptibility.

Subjects having or at risk of having an allergic condition, disorder or disease include subjects with an existing allergic condition or a known or a suspected predisposition towards developing a symptom associated with or caused by an allergic condition. Thus, the subject can have an active chronic allergic condition, disorder or disease, an acute allergic episode, or a latent allergic condition, disorder or disease. Certain allergic conditions, are associated with seasonal or geographical environmental factors. Thus, at risk subjects include those at risk from suffering from a condition based upon a prior personal or family history, and the season or physical location, but which the condition or a symptom associated with the condition may not presently manifest itself in the subject.

A “subject having or at risk of having asthma” refers to a subject suffering from an acute episode of asthma, either a new-onset or a recurrent episode, a subject with a prior history of one or more episodes of asthma, or a subject with a known or suspected predisposition towards developing asthma. A subject having asthma can have active asthma or can be asymptomatic and between acute asthma episodes. A subject having asthma can be suffering from recently acute asthmatic episode (e.g., within minutes or hours of episode onset). A subject having asthma can have a positive skin test, or exhibit one or more symptoms typically associated with acute or chronic asthma, for example, a symptom of allergic asthma. A subject having or at risk of having asthma may be or has been exposed to an allergen, for example, and is at increased risk of suffering from an asthmatic episode due to a predisposition or susceptibility towards an asthmatic episode upon re-exposure to the allergen. Subjects predisposed or susceptible to, exposed to or allergic to these or other allergens are at risk of having asthma and, therefore, are amenable to treatment in accordance with the invention.

At risk subjects also appropriate for treatment in accordance with the invention include subjects exposed to an allergen or are susceptible to having an allergic reaction, or infection or exposure by an agent that is associated with an allergy or allergic reaction. At risk subjects appropriate for treatment in accordance with the invention include subjects having a predisposition towards an allergic reaction, or infection or exposure to an agent that is associated with an allergy or allergic reaction due to a genetic or environmental risk factor. Methods of the invention include subjects contacted with or administered to a binding agent prophylactically.

In the methods of the invention in which a detectable result or beneficial effect is a desired outcome, such as a therapeutic benefit in a subject treated in accordance with the invention, compositions such as binding agents can be administered in sufficient or effective amounts. An “amount sufficient” or “amount effective” includes an amount that can modulate eosinophil migration, chemotaxis or generation.

As used herein, an “amount sufficient” or “amount effective” refers to an amount of a composition or binding agent (e.g., a 5-HT2A receptor agonist or antagonist) that provides, in single or multiple doses, alone or in combination with one or more other (second) compounds or agents (e.g., a drug), treatments or therapeutic regimens, a long or short term detectable response, a desired outcome or beneficial effect in a given subject of any measurable or detectable degree or duration (e.g., for minutes, hours, days, months, years, or cured).

An amount sufficient or an amount effective can but need not be provided in a single administration and can but need not be administered alone (i.e., without a second drug, agent, treatment or therapeutic regimen), or in combination with another compound, agent, treatment or therapeutic regimen. In addition, an amount sufficient or an amount effective need not be sufficient or effective if given in single or multiple doses without a second compound, agent, treatment or therapeutic regimen, since additional doses, amounts or duration above and beyond such doses, or additional drugs, agents, treatment or therapeutic regimens may be included in order to be effective or sufficient in a given subject. Further, an amount sufficient or an amount effective need not be effective in each and every subject, nor a majority of subjects in a given group or population. Thus, an amount sufficient or an amount effective means sufficiency or effectiveness in a particular subject, not a group or the general population. As is typical for such methods, some subjects will exhibit a greater or less response to a method of the invention, including treatment/therapy.

Reducing, inhibiting decreasing, eliminating, delaying, halting or preventing a progression or worsening or an adverse symptom of the condition, disorder or disease is a satisfactory outcome. The dose amount, frequency or duration may be proportionally increased or reduced, as indicated by the status of the condition, disorder or disease being treated, or any adverse side effects of the treatment or therapy. Dose amounts, frequencies or duration also considered sufficient and effective are those that result in a reduction of the use of another drug, agent, treatment or therapeutic regimen or protocol. For example, a 5-HT2A receptor agonist or antagonist is considered as having a beneficial or therapeutic effect if contact, administration or delivery in vivo results in the use of a lesser amount, frequency or duration of another drug, agent, treatment or therapeutic regimen or protocol to treat the condition, disorder or disease, or an adverse symptom thereof.

An “amount sufficient” or “amount effective” includes reducing, preventing, delaying or inhibiting onset, reducing, inhibiting, delaying, preventing or halting the progression or worsening of, reducing, relieving, alleviating the severity, frequency, duration, susceptibility or probability of one or more adverse or undesirable symptoms associated with the condition, disorder or disease of the subject. In addition, hastening a subject's recovery from one or more adverse or undesirable symptoms associated with the condition, disorder or disease is considered to be an amount sufficient or effective. Various beneficial effects and indicia of therapeutic benefit are as set forth herein and are known to the skilled artisan.

An “amount sufficient” or “amount effective,” in the appropriate context, can refer to therapeutic or prophylactic amounts. Therapeutically or prophylactically sufficient or effective amounts mean an amount that detectably improves the condition, disorder or disease, such as an inflammatory, non-allergic or allergic condition, disorder or disease, as assessed by one or more objective or subjective clinical endpoints appropriate for the condition, disorder or disease.

In accordance with the invention, there are provided methods which provide a beneficial effect, such as a therapeutic benefit, to a subject. In one embodiment, a method includes administering an amount of 5-HT2A receptor antagonist sufficient to provide a therapeutic benefit or beneficial effect to a subject. In one aspect, a method reduces the probability, susceptibility, severity, frequency, duration or prevents an acute asthmatic episode (e.g., associated with allergic or non-allergic asthma) in the subject. In another aspect, a method increases, stimulates, enhances, induces or promotes airway-dilation in the subject. In an additional aspect, a method reduces the probability, susceptibility, severity, frequency, duration or prevents or eliminates airway-constriction the subject. In a further aspect, a method is sufficient to reduce progression, severity, frequency, duration, susceptibility, probability, halt, eliminate or prevent one or more adverse physiological or psychological symptoms associated with asthma (allergic or non-allergic). Further aspects include delivery of 5-HT2A receptor antagonist to the lungs or airways.

Sufficiency or effectiveness of a particular treatment can be ascertained by various clinical indicia and endpoints. For example, in order to ascertain an improvement in asthma, an increase in airway dilation, lung function or a reduction in airway constriction, obstruction or narrowing, progression, severity, duration, frequency, susceptibility or probability of one or more symptoms of asthma. An “amount sufficient” or “amount effective” to treat asthma is therefore an amount that provides an objective or subjective reduction or improvement in progression, severity, frequency, susceptibility or probability of lung or airway inflammation, lung or airway tissue damage, shortness of breath, wheezing, coughing, chest-tightness, chest pain, increased heart rate, runny nose, airway or broncho-constriction, -obstruction or narrowing, decreased lung capacity, acute asthmatic episodes and nighttime awakenings. Thus, a reduction, decrease, inhibition, delay, halt, prevention or elimination of one or more adverse symptoms (e.g., shortness of breath, wheezing, coughing, chest-tightness, chest pain, increased heart rate, runny nose, acute asthmatic episodes and nighttime awakenings) can be used as a measure of sufficiency or effectiveness.

A method to determine an improvement in lung or pulmonary function is to measure the forced expiratory volume in one second (FEV₁) an increase of which indicates an improvement. Spirometry is a test which measures the amount and rate at which air can pass through airways. Airway narrowing due to inflammation restricts air flow through the airways, which is detected by changed spirometry values. Exercise challenge and methacholine inhalation tests are also used to evaluate airway narrowing or constriction. Yet another method to determine an improvement is to measure serum IgE in a subject. A reduction in serum or bronchoalveolar lavage (BAL) fluid IgE is an objective measure of treatment efficacy. Various additional methods are known in the art for detecting improvement in lung or pulmonary function.

An “amount sufficient” or “amount effective” also includes an amount that, when used in combination with another binding agent, drug, or treatment or therapeutic regimen, reduces the dosage frequency, dosage amount, or an adverse symptom or side effect of the other binding agent, drug or treatment or therapeutic regimen, or eliminates the need for the other binding agent, drug or treatment or therapeutic regimen. For example, an “amount sufficient” or “amount effective” of a 5-HT2A receptor antagonist could result in a reduction in the dosage frequency or dosage amount of a steroid, antihistamine, beta adrenergic agonist, anticholinergic, methylxanthine, anti-IgE, anti-leukotriene, anti-beta2 integrin, anti-CCR3 antagonist, or anti-selectin required to achieve the same clinical endpoint.

The terms “treat,” “therapy” and grammatical variations thereof when used in reference to a method means the method provides an objective or subjective (perceived) improvement in a subjects' condition, disorder or disease, or an adverse symptom associated with the condition, disorder or disease. Non-limiting examples of an improvement can therefore reduce or decrease the probability, susceptibility or likelihood that the subject so treated will manifest one or more symptoms of the condition, disorder or disease. Additional symptoms and physiological or psychological responses caused by or associated with conditions, disorders or diseases associated with, for example, asthma are set forth herein and known in the art and, therefore, improvements in these and other adverse symptoms or physiological or psychological responses can also be included in the methods of the invention.

Methods of the invention therefore include providing a detectable or measurable beneficial effect or therapeutic benefit to a subject, or any objective or subjective transient or temporary, or longer-term improvement (e.g., cure) in the condition. Thus, a satisfactory clinical end point is achieved when there is an incremental improvement in the subjects condition or a partial reduction in the severity, frequency, duration or progression of one or more associated adverse symptoms or complications or inhibition, reduction, elimination, prevention or reversal of one or more of the physiological, biochemical or cellular manifestations or characteristics of the condition, disorder or disease. A therapeutic benefit or improvement (“ameliorate” is used synonymously) therefore need not be complete ablation of any or all adverse symptoms or complications associated with the condition, disorder or disease but is any measurable or detectable objectively or subjectively meaningful improvement in the condition, disorder or disease. For example, inhibiting a worsening or progression of the condition, disorder or disease, or an associated symptom (e.g., slowing or stabilizing one or more symptoms, complications or physiological or psychological effects or responses), even if only for a few days, weeks or months, even if complete ablation of the condition, disorder or disease, or an associated adverse symptom is not achieved is considered to be beneficial effect.

Prophylactic methods are included. “Prophylaxis” and grammatical variations thereof mean a method in accordance with the invention in which contact, administration or in vivo delivery to a subject is prior to manifestation or onset of a condition, disorder or disease (or an associated symptom or physiological or psychological response), such that it can eliminate, prevent, inhibit, decrease or reduce the probability, susceptibility or frequency of having a condition, disorder or disease, or an associated symptom. Target subject's for prophylaxis can be one of increased risk (probability or susceptibility) of contracting the condition, disorder or disease, or an associated symptom, or recurrence of a previously diagnosed condition, disorder or disease, or an associated symptom, as set forth herein and known in the art.

Any compound or agent.(e.g., drug), therapy or treatment having a beneficial, additive, synergistic or complementary activity or effect (beneficial or therapeutic) can be used in combination with a binding agent in accordance with the invention. A “second compound” or “second agent” refers to any compound or agent (e.g., drug) that is not the first compound or agent of the recited composition, e.g., if a first drug or agent is a particular 5-HT2A receptor agonist or antagonist, then a second drug or agent is different from the first 5-HT2A receptor agonist or antagonist. The second compound or agent can be a salt, fee base, ester, derivative, racemate (+ or − enantiomer mixture), or prodrug of the first compound or agent, for example, a 4-piperidine-methanol or N-aralkyl-piperidine-methanol derivative such as (+)-α-(2,3-dimethoxyphenyl)-1-[2-(4-fluorophenyl)ethyl]-4-piperidinemethanol (MDL-100,907); α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl)}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; and α-(2,3-dimethoxyphenyl)-1-[2-(4-(2′,2′,2′-trifluoroethoxyphenyl)ethyl]-4-piperidine methanol, or salt, free base, ester, derivative, racemate (+ or − enantiomer mixture), or prodrug thereof. The second compound or agent can but need not be selective, for example, for binding to 5-HT2A receptor.

In accordance with the invention there are provided methods in which a second compound or agent (e.g., drug) is administered to the subject. In one embodiment, a second compound or agent (e.g., drug) is administered to the subject prior to, with or following contacting or administering a 5-HT2A receptor agonist or antagonist.

Methods of the invention therefore include combination therapies and treatments. Examples of such combination therapies include separate or pooled compounds or binding agents of 5-HT2A (e.g., pooled agonists or antagonists, compounds or agents that modulate activity or expression), each having a different activity, affinity, selectivity (specificity) or efficacy in a method as set forth herein or known in the art. Accordingly, combination compositions, therapies and treatments are provided, as well as methods of using such combinations, therapies and treatments in conjunction with the methods of the invention. Contact, administration or in vivo delivery of a compound or agent, such as a binding agent, or practice of a therapy or treatment, can occur prior to, in conjunction with or following a method of the invention.

Non-limiting examples of functional classes of compounds and agents useful as a second compound or agent (e.g., drug) include anti-inflammatory, anti-asthmatic, airway dilators (e.g., xanthine drugs such as methylxanthines, which are broncho-dilators) and anti-allergy drugs. Additional non-limiting examples of compounds and agents useful for employing in the invention, for example to treat an allergic condition, disorder or disease (e.g., asthma, allergic rhinitis) include hormones, such as steroids (e.g., glucocorticoids); antihistamines; beta adrenergic agonists; anticholinergics; methylxanthines; anti-IgE; anti-leukotrienes; anti-beta2 integrins; anti-alpha-4 integrins; H1-receptor antagonists; anti-CCR3 antagonists; and anti-selectins.

Specific non-limiting examples of glucocorticoids include dexamethasone, triamcinolone acetonide (AZMACORT®), beclomethasone, dipropionate (VANCERIL®), flunisolide (AEROBID®), fluticasone propionate (FLOVENT®), prednisone, methylprednisolone and mometasone furoate (ASMANEX®, TWISTHALER®). Specific non-limiting examples of antihistamines include chlorcyclizine, chlorpheniramine, triprolidine (ACTIFED®), diphenhydramine hydrochloride (BENADRYL®), fexofenadine hydrochloride (ALLEGRA®), hydroxyzine hydrochloride (ATARAX®), loratadine (CLARITIN®), promethazine hydrochloride (PHENERGAN®), pyrilamine; and anti-IgE omalizumab (XOLAIR®). Specific non-limiting example of beta adrenergic agonists include albuterol (VENTOLIN®; PROVENTIL®), Xopenex®, (S)-isomer subtracted from racemic albuterol (Sepracor Inc.), pirbuterol, epinephrine, racepinephrine, adrenaline, isoproterenol, salmeterol (Serevent®), metaproterenol (ALUPENT®), bitolterol (Tornalate®), fenoterol (BEROTEC®), formoterol (Foradil®), isoetharine, procaterol, β2-adrenoceptor and terbutaline (BRETHINE®, LAMISIL®). A specific non-limiting example of an anticholinergic (cholinergic receptor antagonist) includes ipratropium bromide (ATROVENT®) and tiotropium. Specific non-limiting examples of methylxanthines include theophylline, aminophylline, theobromine, cromolyn (Intal®) and nedocromil (Fisons). A specific non-limiting example of an anti-IgE is omalizumab (XOLAIR®). Specific non-limiting examples of anti-leukotrienes (leukotriene inhibitors) include cysteinyl-leukotriene (Cys-LT), Singulair® and Accolate®.

Anti-inflammatory agents useful for employing in the methods include cytokines and chemokines. Particular non-limiting examples of cytokines include anti-inflammatory cytokines such as IL-4 and IL-10. Anti-cytokines and anti-chemokines, such as antibodies that bind to pro-inflammatory cytokines, TNFα, IFNγ, IL1, IL-2, IL-6, etc., as well as anti-Th2 cytokines such as IL-5, IL-13, etc., can be employed in the methods.

Additional functional classes of compounds and agents useful as a second compound or agent (e.g., drug) include selective or non-selective potassium channel activators (bronchodilatators); muscarinic M3 receptor antagonists; M2 receptor agonists; opioid receptor agonists (inhibit release of sensory neuropeptides); H3-receptor agonists (inhibit acetylcholine release); phospholipase A2 inhibitors; 5-lipoxygenase inhibitors; 5-lipoxygenase activating protein (FLAP) inhibitors; phosphodiesterase inhibitors; immunomodulating agents (Ciclosporine); antibody against adhesion molecules; and antagonists of tachykinins (e.g., Substance P or neurokinin).

In various particular embodiments, a subject treated in accordance with the invention, such as a subject in need of treatment for a condition, disorder or disease herein, has not previously been administered a 5-HT2A receptor antagonist (e.g., 4-piperidine-methanol or N-aralkyl-piperidine-methanol derivative, (+)-α-(2,3-dimethoxyphenyl)-1-[2-(4-fluorophenyl)ethyl]-4-piperidinemethanol (MDL-100,907) or a salt, free base, ester, derivative, racemate (+ or − enantiomer mixture), or prodrug thereof for treatment of an acute or chronic neurological or psychological disorder. In various aspects, the neurological or psychological disorder is anxiety, anorexia nervosa, insomnia, sleep apnea, obsessive compulsive disorder, psychosis (e.g., shared or substance-induced delusions, hallucinations, or illusions), bipolar disorder, depression, dysthymia, schiozophrenia, mania, substance abuse (e.g., chronic or acute alcohol, nicotine, a narcotic, an opiate, a stimulant, cocaine, amphetamine, methamphetamine or dextroamphetamine abuse), or migraines.

In additional particular embodiments, a subject treated in accordance with the invention, such as a subject in need of treatment for a condition, disorder or disease herein, has not previously been administered a 5-HT2A receptor antagonist (e.g., 4-piperidine-methanol or N-aralkyl-piperidine-methanol derivative, (+)-α-(2,3-dimethoxyphenyl)-1-[2-(4-fluorophenyl)ethyl]-4-piperidinemethanol (MDL-100,907) or a salt, free base, ester, derivative, racemate (+ or − enantiomer mixture), or prodrug thereof for treatment of an acute or chronic cardiac disorder (myocardial infarction, ischemia, stable or variant angina, coronary vasospasms, or coronary arrhythmia, such as atrial tachycardia, atrial flutter, atrial fibrillation, ventricular tachycardia or ventricular fibrillation), vascular disorder (e.g., peripheral vascular disease, glaucoma, intermittent claudication, peripheral vasospasms, a thrombotic illness, an embolitic illness, stroke) or hypertension.

In further particular embodiments, a subject treated in accordance with the invention, such as a subject in need of treatment for a condition, disorder or disease herein, has not previously been administered a 5-HT2A receptor antagonist (e.g., 4-piperidine-methanol or N-aralkyl-piperidine-methanol derivative, (+)-α-(2,3-dimethoxyphenyl)-1-[2-(4-fluorophenyl)ethyl]-4-piperidinemethanol (MDL-100,907) or a salt, free base, ester, derivative, racemate (+ or − enantiomer mixture), or prodrug thereof for treatment of fibromyalgia, Raynaud's phenomenon, or for an anesthetic or analgesic treatment.

In additional particular embodiments, a subject treated in accordance with the invention, such as a subject in need of treatment for a condition, disorder or disease herein, has not previously been administered a 5-HT2A receptor antagonist (e.g., 4-piperidine-methanol or N-aralkyl-piperidine-methanol derivative, (+)-α-(2,3-dimethoxyphenyl)-1-[2-(4-fluorophenyl)ethyl]-4-piperidinemethanol (MDL-100,907) or a salt, free base, ester, derivative, racemate (+ or − enantiomer mixture), or prodrug thereof for treatment of an acute or chronic extrapyramidal side effect (EPSE) associated with a neuroleptic drug (e.g., dysphoria, akathisia, a cognitive impairment, parkinsonian-like syndrome, tremors, or loss of motivation).

Compositions including binding agents can be included in a pharmaceutically acceptable carrier (excipient, diluent, vehicle or filling agent) for administration to a subject. The terms “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Exemplary antifungals include agents such as benzoic acid, undecylenic alkanolamide, ciclopiroxolamine, polyenes, imidazoles, allylamine, thicarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, ketoconazole, undecylenate, silver sulfadiazine, undecylenic acid, undecylenic alkanolamide and Carbol-Fuchsin.

The pH can be adjusted by use or addition of pharmacologically acceptable acids or bases. Examples of inorganic acids include: hydrochloric acid, hydrobromic acid, nitric acid, sulfuric acid, and/or phosphoric acid. Examples of organic acids are: ascorbic acid, citric acid, malic acid, tartaric acid, maleic acid, succinic acid, fumaric acid, acetic acid, formic acid and/or propionic acid, etc. Acids which form an acid addition salt with the active ingredient may also be used. Examples of bases include alkali metal hydroxides and alkali metal carbonates. If such bases are used, the resulting salts which are contained in the pharmaceutical formulation, are typically compatible with the acid. If desired, mixtures of acids or bases may also be used.

Pharmaceutical compositions can optionally be formulated to be compatible with a particular route of administration. Thus, pharmaceutical compositions include carriers (excipients, diluents, vehicles or filling agents) suitable for administration by various routes and delivery to targets, locally, regionally or systemically.

Exemplary routes of administration for contact or in vivo delivery which a composition can optionally be formulated include respiratory system (nasal, inhalation, respiration, intubation, intrapulmonary instillation), oral, buccal, intrapulmonary, rectal, intrauterine, intradermal, topical, dermal, parenteral, sublingual, subcutaneous, intravascular, intrathecal, intraarticular, intracavity, transdermal, iontophoretic, intraocular, ophthalmic, optical, intravenous, intramuscular, intraglandular, intraorgan, intralymphatic.

For delivery across the blood-brain or blood-spinal cord barrier of a subject, oral administration of lipid soluble (lipophilic) molecules or lipidation of water soluble molecules have been used. For example, an oral dose of M100,907 (20 mg daily) induced a very high 5HT(2A) receptor occupancy in the frontal cortex of two clinical subjects. Thus, for M100,907 compositions in which it is desired to achieve delivery across the blood-brain or blood-spinal cord barrier, oral administration is suitable. Intra-cranial and in some aspects, oral administration of M100,907, would not be used if delivery across blood-brain or blood-spinal cord barrier was not desired.

Nasal and instillation formulations typically include aqueous solutions of active ingredient (compounds or agents) optionally with one or more preservative or isotonic agents. Such formulations are typically adjusted to a pH and isotonic state compatible with nasal mucous membranes. A solvent may include only water, or it may be a mixture of water and one or more other components (e.g., ethanol). Typically, the maximum ethanol is up to about 70-75% % by volume. The remaining volume may consist of water or one or more other solvents in various proportions.

Formulations that include respirable or inhalable liquid or solid particles of the active ingredient, (e.g., compound, binding agent) can have particles of a size sufficiently small to pass through the mouth and larynx upon inhalation and continue into the airways of the lungs (e.g., bronchi and alveoli). Particles typically range in size from about 0.05, about 0.1, about 0.5, about 1, about 2 to about 4, about 6, about 8, about 10 microns in diameter. Particles of non-respirable size can be included in an aerosol or spray to deposit in the throat. For nasal administration or intrapulmonary instillation, a particle size in the range of about 8, about 10, about 20, about 25 to about 35, about 50, about 100, about 150, about 250, about 500 μm (diameter) is typical for retention in nasal cavity or for instillation into lung.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof.

Compounds including binding agents either alone or in combination with a pharmaceutically acceptable carrier, second compound, etc. can be administered into the respiratory system of a subject by inhalation, respiration, intubation, or intrapulmonary instillation (into the lungs), for example. Respiratory administration can be achieved using an aerosol or spray of a gas, liquid or powdered nasal, intrapulmonary, respirable or inhalable in a particle form. The particles include the compound or binding agent, and optionally any other component (e.g., second compound), and are administered or delivered to the subject by inhalation, by nasal administration or instillation into the airways or the lung.

Administration to airways can be accomplished using an article of manufacture, such as container with or without an aerosol. Liquid formulations may be squirted into the respiratory system (e.g., nose) and the lung from a container by pressure or using an aerosol propellant or a spray device or delivery system. Administration can be passive or it can be assisted by a pressurized delivery system or device. An aerosol, delivery system or device can include a pressurized container containing liquid, gas or dry powder.

An “aerosol formulation” refers to a preparation that includes droplets or particles of active ingredient (e.g., compound, binding agent) suitable for delivery to respiratory system (e.g., lung, airway, nasal and sinus epithelium). The aerosol formulation can include a sufficient or effective amount of a compound or agent and a pharmaceutically acceptable carrier, optionally a propellant, in a container or aerosol or spray device or delivery system. Aerosol formulations can deliver high concentrations into airways with relatively low systemic absorption, and include for example nasal sprays, inhalation solutions, inhalation suspensions, and inhalation sprays. Nasal sprays typically contain active ingredient dissolved or suspended in solution or in an excipient, in nonpressurized dispensers that deliver a metered dose of the ingredient.

For aerosol delivery, pH of the formulation is typically between 5.0 and 7.0. If the aerosol is too acidic or basic, it can cause bronchospasm and cough. The tolerized pH range is relative and depends on a patient's tolerance: some patients tolerate a mildly acidic aerosol, which in others will cause bronchospasm. Typically, an aerosol formulation having a pH less than 4.5 induces bronchospasm.

Compositions including compounds and binding agents can be formulated in a dry powder for delivery into the endobronchial space. Dry powder formulations provide stability, high volume delivery per puff, and low susceptibility to microbial growth. Dry powder formulations typically are stable at ambient temperature, and have a physiologically acceptable pH of 4.0-7.5. Dry powder formulations are convenient because they do not require any further handling, such as dilution, prior to administration. Depending on delivery device efficiency, effective dry powder dosage levels typically fall in the range of about 10 to about 100 mg. Dry powder formulations can be used directly in metered dose or dry powder inhalers.

Aerosol and spray delivery systems and devices, also referred to as “aerosol generators” and “spray generators” are known in the art and include metered dose inhalers (MDI), nebulizers (ultrasonic, electronic and other nebulizers), nasal sprayers and dry powder inhalers.

MDIs typically include an actuator, a metering valve, and a container that holds a suspension or solution, propellant, and surfactant (e.g., oleic acid, sorbitan trioleate, lecithin). The container may be pressurized or not, but typically it is either squeezed to dispense the ingredient, or has an actuator connected to a metering valve so that activation of the actuator causes a predetermined amount to be dispensed from the container in the form of an aerosol, which is inhaled by the subject. MDIs typically use liquid propellant. Typically, metered-dose aerosol inhalers create droplets that are 15 to 30 microns in diameter. Currently, MDI technology is optimized to deliver masses of 1 microgram to 10 mg of a therapeutic.

Nebulizers, also referred to as atomizers, are devices that turn medication into a fine mist inhalable by a subject through a face mask that covers the mouth and nose. Nebulizers provide small droplets and high mass output which can be delivered to upper and lower respiratory airways. Typically, nebulizers create droplets down to about 1 micron in diameter. Doses administered by nebulizers are typically larger than doses administered by MDIs.

Nebulizers include air-jet and ultrasonic nebulizers, in fluid connection with a reservoir containing disposed therein a solution or suspension of active ingredient. Nebulizers (air-jet, ultrasonic or electronic) are typically used for acute care of nonambulatory patients and in infants and children. Airjet nebulizers are relatively large but considered portable because of the availability of small compressed air pumps. Ultrasonic and electronic nebulizers are typically more portable because they usually do not require a source of compressed air. An example of an airjet nebulizer is the NE-C25 CompAir XLT Compressor Nebulizer System (Omron® Healthcare). Examples of ultrasonic nebulizers include the Zewa Portable Ultrasonic Nebulizer (Zewa, Inc.); the MabisMist II Ultrasonic Nebulizer (Mabis Healthcare, Inc.); and the MICROAir Ultrasonic Nebulizer (Omron® Healthcare). An example of an electronic nebulizer is the Micro-Air® Electronic Nebulizer with V.M.T. (Omron® Healthcare). Modified nebulizers can have the addition of a one-way flow valve (e.g., Pari LC Plus™, Pari Respiratory Equipment, Inc.), which delivers up to 20% more drug than unmodified nebulizers.

Components of the nebulizer are typically made of a material suitable for their intended function. The housing of the nebulizer and, if the function allows, other parts can be made of plastic (PVC, Polycarbonate, polystyrene, polypropylene, polybutylene, etc.). Plastic can be formed by injection molding. For medical applications, physiologically acceptable materials are used.

Dry-powder inhalers (DPI) can be used to deliver the compounds or agents, either alone or in combination with a pharmaceutically acceptable carrier, second compound, etc. Dry powder inhalers deliver active ingredient to airways and lungs while the subject inhales through the device. DPIs typically do not contain propellants or any other ingredients, only the medication, but may optionally include other components. DPIs are typically breath-activated, but may involve air or gas pressure to assist delivery. For breath-activated DPIs, a subject need not coordinate breathing with the activation of the inhaler.

There are two major DPI design classes. The first is a device-metering design in which a reservoir of drug is stored within the device and the subject ‘loads’ a dose of the device into the inhalation chamber, and the inspiratory flow of the patient accelerates the powder out of the device and into the oral cavity. The second type of DPI may also employ an air source, a gas source, or electrostatics, in order to deliver the active ingredient. Non-limiting examples of DPIs include Spinhaler® (Rhone-Poulenc Rorer Pharmaceuticals, Collegeville, Pa.), Inhalator® (Boehringer Ingelheim, Ingelheim, Germany), Rotahaler® (GlaxoSmithKline), Turbulaler® (Astra Draco Pharmaceuticals, Lund, Sweden) and Accuhaler (GlaxoSmithKline).

An aerosol, delivery system or device can include a propellant. Exemplary propellants include chlorofluorocarbons (e.g., trichlorofluoromethane, dichlorodifluoromethane, dichlorotetrafluoromethane, CFC-11, CFC-12) and the non-d chlorofluorocarbons, HFC-134A and HFC-227. Suitable fluorocarbon (HFA) propellants are known in the art and include, for example, HFA134a (1,1,1,2-tetrafluoroethane), HFA227 (1,1,1,2,3,3,3-heptafluoro-n-propane) and mixtures of HFA134a and HFA227.

Pharmaceutical acceptable compositions can be formulated so that significant traversal of a compound or agent across the blood-brain or blood-spinal cord barrier does not occur. For example, water soluble non-lipophilic pharmaceutically acceptable carriers typically do not efficiently traverse the blood-brain or blood-spinal cord barrier. In certain embodiments of the invention, it is desired that amounts of a 5-HT2A receptor antagonist that traverses (crosses) the blood-brain or blood-spinal cord barrier be less than the amount sufficient or effective for treating a condition as set forth herein. Thus, water soluble non-lipophilic pharmaceutically acceptable compositions can be employed in order to minimize traversal across the blood-brain or blood-spinal cord barrier.

Routes of administration can also be used so that significant traversal of a compound or agent across the blood-brain or blood-spinal cord barrier does not occur. Of course, traversal of the blood-brain or blood-spinal cord barrier will also depend on the nature of the compound or pharmaceutical formulation used, e.g., non-lipophilic. Particular non-limiting examples include buccal, intrapulmonary, intradermal, topical, dermal, parenteral, sublingual, nasal, subcutaneous, intrathecal, inhalable, respirable, intraarticular, transdermal, iontophoretic, ophthalmic, optical, intramuscular, and intraglandular route.

Methods for measuring transport across the blood-brain or blood-spinal cord barrier are known in the art. For example, computer models can predict transport of small molecules across blood-brain barrier based upon physico-chemical properties of the molecule, such as molecular weight, hydrogen bonding, lipid solubility, and polar surface area. In, vitro assays in which brain capillary endothelial cells are grown on porous membranes and placed in diffusion chambers can be used for measurement of drug transport across the monolayer. Drug transport into brain can measured in vivo with an intravenous injection technique, providing the time interval used to monitor drug transport (e.g. 15-60 seconds) is sufficiently short to eliminate or minimize metabolism artifacts. Two such in vivo, techniques are the internal carotid artery perfusion (ICAP) method and the common carotid artery single injection or brain uptake index (BUI) method.

Exemplary conditions for which significant traversal of a compound or agent (e.g., a 5-HT2A receptor antagonist) across the blood-brain or blood-spinal cord barrier is not desired at levels sufficient or effective for treatment include an acute or chronic neurological or psychological disorder (e.g., anxiety, anorexia nervosa, insomnia, sleep apnea, obsessive compulsive disorder, psychosis, such as shared or substance-induced delusions, hallucinations, or illusions, bipolar disorder, depression, dysthymia, schiozophrenia, mania, substance abuse, such as chronic or acute alcohol, nicotine, a narcotic, an opiate, a stimulant, cocaine, amphetamine, methamphetamine or dextroamphetamine abuse, or migraines; cardiac disorder (e.g., myocardial infarction, ischemia, stable or variant angina, coronary vasospasms, or coronary arrhythmia, such as atrial tachycardia, atrial flutter, atrial fibrillation, ventricular tachycardia or ventricular fibrillation), vascular disorder (e.g., peripheral vascular disease, glaucoma, intermittent claudication, peripheral vasospasms, a thrombotic illness, an embolitic illness, stroke) or hypertension; fibromyalgia, Raynaud's phenomenon, an anesthetic or analgesic treatment; an extrapyramidal side effect (EPSE) associated with a neuroleptic drug (e.g., dysphoria, akathisia, a cognitive impairment, parkinsonian-like syndrome, tremors, or loss of motivation). Accordingly, pharmaceutically acceptable compositions that do not efficiently traverse the blood-brain or blood-spinal cord barrier can be employed in the invention, as appropriate.

Pharmaceutical compositions and delivery systems appropriate for compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index, (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1,980), R. L. Juliano, ed., Oxford, N.Y., pp.253-315)

Compounds, binding agents and pharmaceutical compositions thereof can be packaged in unit dosage form (capsules, troches, cachets, lozenges, or tablets) for ease of administration and uniformity of dosage. “Unit dosage form” as used herein refers to physically discrete units suited as dosages for treatment or therapy. Each unit contains a predetermined quantity of agent in association with the pharmaceutical carrier (excipient, diluent, vehicle or filling agent) which, when administered in one or more doses, is calculated to produce a desired beneficial effect. Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compositions for transdermal administration, such as “patches” adapted to remain in contact with the epidermis of the intended recipient for an extended or brief period of time. The individual unit dosage forms can be included in multi-dose kits or containers.

Dose amounts, frequency and duration for binding agents, including agonists or antagonists, such as 5-HT2A receptor antagonists (e.g., a 4-piperidine-methanol or N-arakyl-piperidine-methanol derivative, such as (+)-α-(2,3-dimethoxyphenyl)-1-[2-(4-fluorophenyl)ethyl]-4-piperidinemethanol salt, free base, ester, derivative, racemate (+ or − enantiomer mixture), or prodrug therof can be can be empirically determined in appropriate animal models. Dose amounts, frequency and duration can also be determined and optimized in human clinical trials.

The dosage amount at which (+)-α-(2,3-dimethoxyphenyl)-1-[2-(4-fluorophenyl)ethyl]-4-piperidinemethanol exhibits 5-HT2A receptor antagonist activity can range from about 0.0001 mg/kg of subject body weight/day to about 1,000.0 mg/kg of subject body weight/day. Of course, doses can be more or less, as appropriate, for example, 0.00001 mg/kg of subject body weight to about 10,000.0 mg/kg of subject body weight, about 0.001 mg/kg, to about 100 mg/kg, about 0.01 mg/kg, to about 10 mg/kg, or about 0.1 mg/kg, to about 1 mg/kg of subject body weight over a given time period, e.g., 1, 2, 3, 4, 5 or more hours, days, weeks, months, years, in single bolus or in divided/metered doses. As a non-limiting example, for treatment of an allergic or non-allergic inflammatory condition, disorder or disease of the airways (e.g., allergic or non-allergic asthma or rhinitis), a subject may be administered in single bolus or in divided/metered doses in the range of about 10 to 50,000 micrograms (“mcg”)/day, 10 to 20,000 mcg/day, 10 to 10,000 mcg/day, 25-1,000 mcg/day, 25 to 400 mcg/day, 25-200 mcg/day, 25-100 mcg/day or 25-50 mcg/day, which can be adjusted to be greater or less according to the weight of the subject, e.g., per pound, kilogram, etc.

Compounds and agents (e.g., 5-HT2A receptor agonist or antagonist) can be administered to a subject at any frequency, as a single bolus or in divided/metered doses, one, two, three, four or more times over a given time period, e.g., per hour, day, week, month or year. Exemplary dosage frequencies for airway or lung conditions, disorders or diseases, such as asthma can vary, but are typically from 1-7 times, 1-5 times, 1-3 times, 2-times or once, daily, weekly or monthly, to reduce, inhibit, decrease, delay, prevent, halt or eliminate progression, severity, frequency, duration, or probability of one or more adverse symptoms of the conditions, disorders or diseases, as set forth herein or that would be apparent to one skilled in the art. Timing of contact, administration or in vivo delivery can be dictated by the condition, disorder or disease to be treated. For example, an amount can be administered to the subject substantially contemporaneously with, or within about 1-60 minutes or hours of the onset of a symptom associated with undesirable or abnormal eosinophil migration, chemotaxis or generation (e.g., non-allergic asthma, allergic asthma, an asthmatic episode or airway-constriction, -narrowing or -obstruction).

Dosage amount, frequency or duration can be increased, if necessary, or reduced, for example, once control of the condition, disorder or disease is achieved, dose amounts, frequency or duration can be reduced. Other conditions, disorders or diseases of the airways and lungs can be similarly treated, dosing amount, frequency or duration reduced, when adequate control of the condition, disorder or disease is achieved.

Of course, the dosage amount, frequency and duration can vary depending upon the judgment of the skilled artisan which will consider various factors such as whether the treatment is prophylactic or therapeutic, the type or severity of the condition, disorder or disease, the associated symptom to be treated, the clinical endpoint(s) desired such as the type and duration of beneficial or therapeutic effect. Additional non-limiting factors to consider in determining appropriate dosage amounts, frequency, and duration include previous or simultaneous treatments, potential adverse systemic, regional or local side effects, the individual subject (e.g., general health, age, gender, race, bioavailability), condition of the subject such as other disorders or diseases present and other treatments or therapies that the subject has or is undergoing (e.g., medical history). The skilled artisan will appreciate the factors that may influence the dosage, frequency and duration required to provide an amount sufficient to provide a subject with a beneficial effect, such as a therapeutic benefit.

The invention provides kits including compositions (e.g., 5-HT2A receptor agonists or antagonists) suitable for practicing the methods, treatment protocols or therapeutic regimes herein, and suitable packing material. In one embodiment, a kit includes a 5-HT2A receptor antagonist, and instructions for administering said 5-HT2A receptor antagonist to the lungs or airways of a subject. In another embodiment, a kit includes a 5-HT2A receptor antagonist, an article of manufacture for delivery of the antagonist to the lungs or airways, and instructions for administering said 5-HT2A receptor antagonist to the lungs or airways of a subject. In various aspects, a kit includes a 5-HT2A receptor antagonist selected from (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; and α-(2,3-dimethoxyphenyl)-1-[2-(4-(2′,2′,2′-trifluoroethoxyphenyl)ethyl]-4-piperidine methanol, or a salt, free base, ester, derivative, racemate or prodrug thereof. In additional aspects, a kit includes a second compound (e.g., drug, such as an anti-inflammatory, anti-asthmatic, anti-allergy drug; hormone or a steroid such as a glucocorticoid; an anti-histamine, anti-leukotriene, anti-IgE, anti-α4 integin, anti-β2 integrin, anti-CCR3 antagonist, β2 agonist, or anti-selectin.

The term “packing material” refers to a physical structure housing a component of the kit. The material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to a ampule, tube or vial containing a kit component. Labels or inserts can additionally include a computer readable medium, such as a disk (e.g., floppy diskette, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein (e.g., the binding agent or pharmaceutical composition), dose amounts, clinical pharmacology of the active agent(s) including mechanism of action, pharmacokinetics and pharmacodynamics. Labels or inserts can include information identifying manufacturer information, lot numbers, manufacture location and date.

Labels or inserts can include information on a condition, disorder or disease for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or therapeutic regimes described herein. Exemplary instructions include, instructions for modulating eosinophil migration; instructions for reducing severity, frequency, duration, progression, susceptibility or probability of one or more conditions, disorders or diseases associated with undesirable or abnormal eosinophil migration, chemotaxis or generation, or one or more symptoms associated with undesirable or abnormal eosinophil migration, chemotaxis or generation, as set forth herein or known in the art.

Labels or inserts can include information on any benefit that a component may provide, such as a therapeutic benefit. For example, a Non-limiting examples of a benefit would be improved breathing, increased airway dilation, A benefit could also include a reduced need (amount, frequency or duration) for other medications, treatment protocols or therapeutic regimes, that the subject may be using or have used for treatment of the condition, disorder or disease.

Labels or inserts can include information on potential adverse side effects, such as warnings to the subject or clinician regarding situations where it would not be appropriate to use a particular composition (e.g., a 5-HT2A receptor agonist or antagonist). For example, adverse side effects are generally more likely to occur at higher dose amounts, frequency or duration of the active agent and, therefore, instructions could include recommendations against higher dose amounts, frequency or duration. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities. Non-limiting examples of adverse side effects include, for example, hypersensitivity, rash, neurological effects such as tachycardia; palpitations; headache; tremor and nervousness.

A kit can contain include a components, such as a device suitable for practicing methods, treatment protocols or therapeutic regimes described herein. The device can be used to contact, administer or for in vivo delivery to a subject. The device can be a container, aerosol or spray generator, (e.g., MDI, nebulizer or DPI), vessel or holder for delivery of a compound or agent (e.g., a 5-HT2A receptor agonist or antagonist) to a subject. A non-limiting example of such a device is metered-dose inhaler (MDI) for oral inhalation, which may be pressurized (see, for example U.S. Pat. No. 6,131,566). Suitable packaging for an MDI is described in WO 2000/37336 A1.

Unless otherwise defined, all technical and scientific terms used herein have: the same meaning as commonly understood by one of ordinary skill in the art to which this invention relates. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “a 5-HT2A agonist” includes a plurality of 5-HT2A agonists; and reference to “a symptom” includes a plurality of symptoms (e.g., adverse or undesirable). Of course, this does not preclude limiting certain embodiments of the invention to specific 5-HT2A agonists or antagonists, particular symptoms, particular conditions, disorders or diseases, particular subjects, etc., using appropriate language.

The invention is generally disclosed herein using affirmative language to describe the numerous embodiments. The invention also specifically includes embodiments in which particular subject matter is excluded, in full or in part, such as substances or, materials, method steps and conditions, protocols, procedures, assays or analysis disclosed herein. As an example, the invention includes affirmatively described embodiments in which specific subject matter disclosed herein is excluded from the affirmatively described embodiments. Thus, even though the invention is generally not expressed herein in terms of what the invention does not include, aspects that are not expressly included in the invention are nevertheless expressly or inherently disclosed herein. Furthermore, the invention includes embodiments which exclude subject matter, expressly or inherently disclosed herein or known in the art that, in view of the subject matter and relevant technology, would be incompatible with one or more embodiments of the invention, or the practice thereof would be against public law.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of various materials and methods.

Eosinophil Isolation: Eosinophils were purified from the peripheral blood of allergic donors (Boehme, et al. J. Immunol. 163:1611 (1999)).

Transwell Chamber Chemotaxis Assay: Eosinophils isolated from different allergic donors (n=4) were pre-incubated with MDL-100,907 (Kehne, et al. Neuropsychopharmacology 15:116 (1996); Sorensen, et al. J Pharmacol Exp Ther 266:684 (1993)), at a concentration of 20 μM for 20 min or with media alone before addition (10⁶/well) to Matrigel-coated (200 μg/ml) Transwell chambers. Culture medium alone or medium containing 5-HT (100 nM) or eotaxin (50 nM) was added to the lower chamber. In certain experiments 5-HT was added to only upper or both upper and lower chambers and incubated at 37° C. for 4 h after which the chambers were removed and the number of migrated cells was quantitated and expressed as a % of total cells added to the well.

Murine model of allergic airway inflammation: Pulmonary eosinophilia was induced in female BALB/c mice. In brief, mice were sensitized by two i.p. injections with 50 μg ovalbumin (OVA) in 200 μl alum (Pierce). Non-sensitized mice received 200 μl of alum alone. Ten days following the second injection, MDL-100,907 was administered (0.5 mg/kg body weight i.p.) to the OVA challenged mice. Fifteen minutes after injection of the inhibitor, mice were exposed to three inhalations (30 min each) of aerosolized OVA (10 mg/ml in 0.9% sterile saline) at 1 h intervals. Non-sensitized mice received an i.p. injection of vehicle and aerosol challenge of saline only. One hour after the last aero-allergen challenge, mice were assessed for airway hyperreactivity/responsiveness (AHR) as described below and subsequently sacrificed and processed for BAL fluid collection to determine eosinophil counts (Broide, et al. Am. J. Respir. Cell Mol. Biol. 18:218 (1998)).

Determination of AHR to methacholine: AHR was assessed after completion of the final OVA inhalation challenges, immediately before the mice were sacrificed using a single chamber whole body plethysmograph obtained from Buxco (Troy, N.Y.), as previously described (Broide, et al. Am. J. Physiol. Lung Cell Mol Physiol. 282:L259 (2002)). In this system, an unrestrained, spontaneously breathing mouse is placed into the main chamber of the plethysmograph, and pressure differences between this chamber and a reference chamber are recorded. In the plethysmograph, mice were exposed for 3 min to nebulized PBS and subsequently to increasing concentrations of nebulized MCh (Sigma, St. Louis, Mo.) in PBS using an Aerosonic ultrasonic nebulizer (DeVilbiss). After each nebulization, recordings were taken for 3 min. The Penh values measured during each 3-min sequence are expressed for each MCh concentration (3-24 mg/ml) (Broide, et al. Am. J. Physiol. Lung Cell Mol Physiol. 282:L259 (2002)).

### Example 2

This example includes a description of several study results.

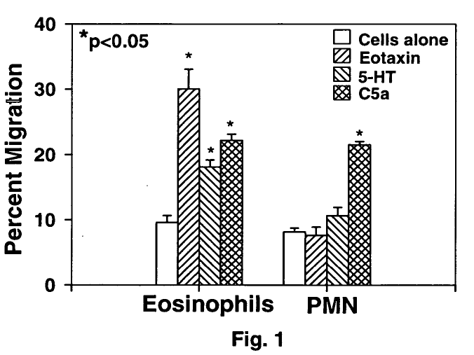

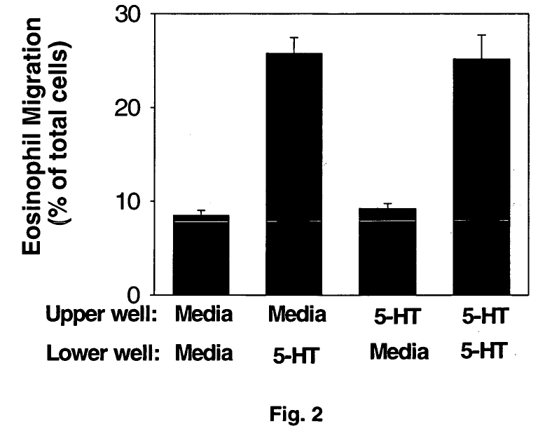

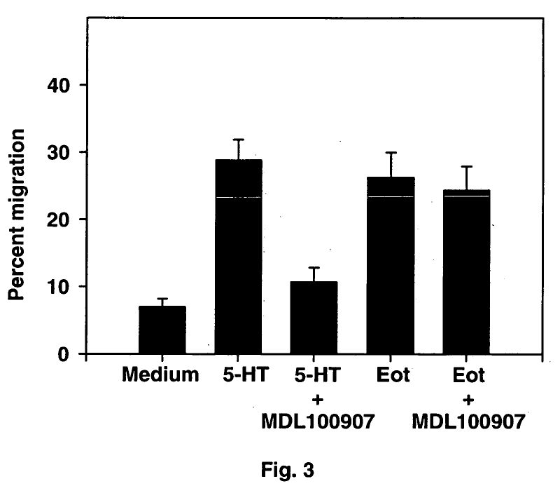

Human eosinophils respond functionally to 5-HT: 5-HT alone was found to induce migration of human eosinophils in a dose-dependent manner, which was maximal at 10⁻⁶ molar (M). Moreover, 5-HT was found to be selective for eosinophils and did not induce chemotaxis of neutrophils, while, C5a (10⁻⁷ M), which was used as a positive control, was found to induce migration of both eosinophils and neutrophils (FIG. 1). It should be pointed out, migration induced by 5-HT was a directed chemotactic response and not chemokinetic (FIG. 2). 5-HT2A receptor antagonist MDL-100,907 inhibits 5-HT-induced human eosinophil chemotaxis in vitro: The effect of MDL-100,907, a highly selective antagonist of 5-HT2A receptor (Kehne, et al. Neuropsychopharmacology 15:116 (1996)), on 5-HT mediated eosinophil chemotaxis was investigated in vitro (FIG. 3). Human eosinophils treated with MDL-100,907 (20 μM) showed a significantly reduced chemotactic response to 5-HT (100 nM) versus untreated eosinophils. In contrast, human eosinophils, either untreated or treated with MDL-100,907 showed a robust migratory response to eotaxin (50 nM), demonstrating MDL-100,907 is not a general inhibitor of eosinophil activity at the dose administered. Rather, the data shows a direct role for the 5-HT2A receptor in 5-HT-mediated chemotaxis of human eosinophils and the ability of MDL-100,907 to block this chemotaxis.

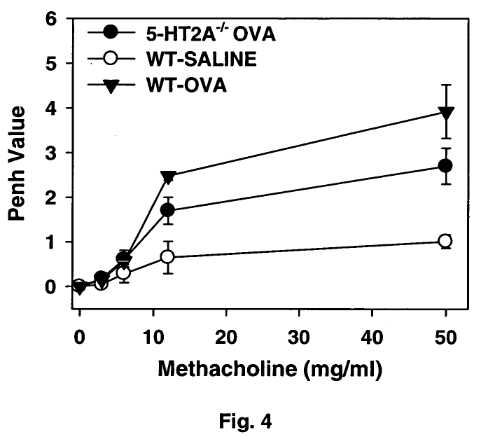

Role of 5-HT2A in eosinophil migration in vitro and AHR using allergen-challenged 5-HT2A^{-/-} (knockout) mice: Studies were conducted with allergen challenged mice to evaluate whether the lack of the 5-HT2A receptor can lead to inhibition of methacholine induced AHR. 5-HT2A^{-/-} C57BL/6 mice and their wild type (WT) counterparts were immunized i.p with 50 μg OVA in alum and boosted 12 days later with an additional dose of OVA (50 μg). On day 22, the two cohorts of immunized mice were challenged with aerosolized OVA at 10 mg/ml for 30 min. The OVA challenge was repeated two more times at 1 h intervals. An additional cohort of wild type mice similarly immunized with alum alone and challenged with nebulized saline served as controls. Three hours after the last aerosol challenge, AHR was measured in the three sets of unrestrained mice using a methacholine challenge and a BUXCO plethysmograph (FIG. 4).

The studies demonstrate that OVA-challenged 5-HT2A^{-/-} mice have reduced methacholine-induced AHR in comparison to OVA-challenged WT litter mates evidencing the role of the 5-HT2A receptor in allergen induced AHR.

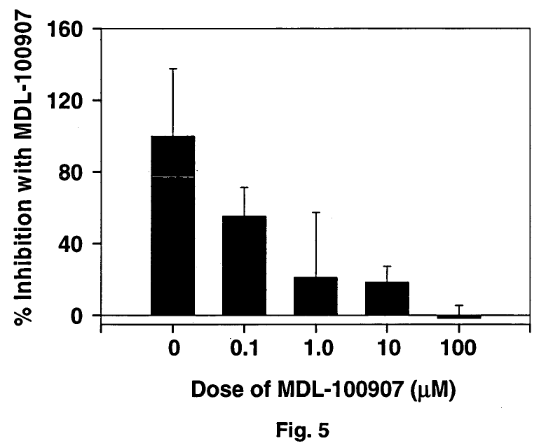

5-HT2A receptor antagonist MDL-100,907 inhibits 5-HT-induced human eosingphil chemotaxis in vitro in a dose dependent manner: The effect of MDL-100,907, a selective antagonist of the 5-HT2A receptor on 5-HT mediated eosinophil chemotaxis was investigated in vitro (FIG. 5). Human eosinophils treated with MDL-100,907 (0-100 μM) showed a significantly reduced chemotactic response to 5-HT (100 nM) versus untreated eosinophils. Robust inhibition of eosinophil chemotaxis was observed at all ranges starting from 0.1 μM to 100 μM demonstrating MDL-100,907 is an effective inhibitor of eosinophil activity at the doses administered.

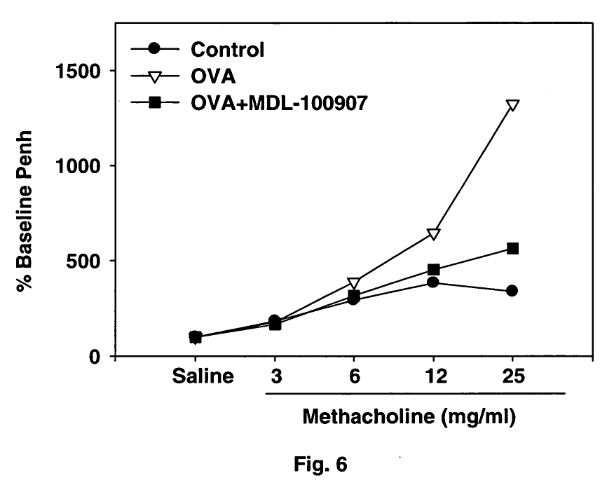

The 5-HT2A receptor antagonist MDL-100,907 inhibits AHR and eosinophil recruitment in the airways of allergen challenged mice: Female. BALB/c mice were immunized intraperitoneally (i.p.) with alum alone (control), 50 μg OVA in alum and boosted 12 days later with an additional dose of OVA (50 μg). On day 22, OVA challenged mice were given MDL-100,907 (0.5 mg/kg body weight i.p.) or an i.p injection of vehicle alone. Fifteen minutes later, mice were challenged with either nebulized OVA (10 mg/ml) or PBS (challenge control) for 30 min. The aerosol challenge was repeated two more times at 1 h intervals. Three hours after the last challenge, AHR was measured in unrestrained mice using BUXCO plethysmography. Thereafter, the mice were euthanized and the bronchoalveolar lavage (BAL) fluid collected. The percentage of the total cells that were eosinophils was quantitated. OVA-challenged MDL-100,907-treated mice exhibited markedly reduced AHR compared to OVA-challenged mice that received PBS alone and not the 5-HT2A antagonist (FIG. 6). Mice challenged with PBS showed minimal AHR and was used as a baseline comparative.

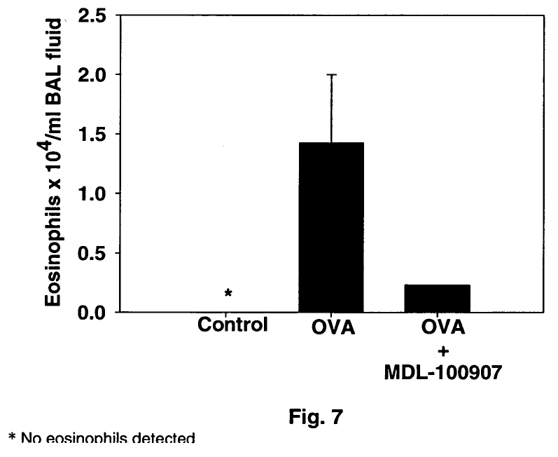

MDL-100,907 also inhibited the number of eosinophils present in the BAL fluid collected from OVA-challenged mice treated with this antagonist compared to OVA-challenged mice that did not receive this compound (FIG. 7). No eosinophils were detected (n.d.) in the BAL fluid of control mice.

Overall, this data demonstrate the in vivo importance of the 5-HT2A receptor to OVA-induced allergic inflammation. Taken together, these studies demonstrate for the first time the ability of MDL-100,907 to inhibit human eosinophil chemotaxis in response to 5-HT in vitro, and importantly block eosinophil recruitment to BAL fluid and AHR in allergen-challenged mice in an animal asthma model. These results indicate that MDL-100,907 can be useful for the treatment of airway allergic inflammation including asthma and other eosinophil mediated diseases.

1. A method of modulating eosinophil migration, chemotaxis or generation, comprising contacting eosinophils with an amount of 5-HT2A receptor agonist or antagonist sufficient to modulate eosinophil migration, chemotaxis or generation.

2. The method of claim 1, wherein the migration, chemotaxis or generation is reduced, decreased, inhibited, delayed, or prevented.

3. The method of claim 1, wherein the migration, chemotaxis or generation is increased, stimulated, enhanced, promoted or induced.

4. The method of claim 1, wherein the antagonist is selective for 5-HT2A receptor.

5. The method of claim 1, wherein the antagonist comprises a 4-piperidine-methanol or N-aralkyl-piperidine-methanol derivative.

6. The method of claim 1, wherein the antagonist comprises (+)-α-(2,3-dimethoxyphenyl)-1-[2-(4-fluorophenyl)ethyl]-4-piperidine methanol (MDL-100,907) or a salt, free base, ester, derivative, racemate (+ or − enantiomer) or prodrug thereof.

7. The method of claim 1, wherein the antagonist comprises α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer) or prodrug thereof.

8. The method of claim 1, wherein the contacting is in vitro or in vivo.

9. The method of claim 8, wherein the in vivo contacting is in a subject that has previously experienced an asthmatic episode or airway- or broncho-constriction or is in need of airway- or broncho-dilation.

10. The method of claim 2, wherein eosinophil migration, chemotaxis or generation is reduced, decreased, inhibited, delayed, halted, or prevented in a pulmonary tissue or organ, gut, or bone marrow.

11. The method of claim 2, wherein eosinophil migration, chemotaxis or generation is reduced, decreased, inhibited delayed, halted, or prevented in lung, airways or respiratory mucosum.

12. A method of reducing or decreasing progression, severity, frequency, duration or probability of one or more symptoms associated with asthma, comprising administering to a subject an amount of 5-HT2A receptor antagonist sufficient to reduce or decrease progression, severity, frequency, duration or probability of the symptom associated with asthma.

13. The method of claim 12, wherein the asthma is caused by an allergen or by exercise.

14. The method of claim 12, wherein the symptom is selected from lung, airways or respiratory mucosum inflammation or tissue damage, shortness of breath, wheezing, coughing, chest-tightness, chest pain, increased heart rate, runny nose, airway-constriction, decreased lung capacity, and an acute asthmatic episode.

15. A method of treating a subject having or at risk of having a condition associated with undesirable or abnormal eosinophil migration, chemotaxis or generation, comprising administering to a subject an amount of 5-HT2A receptor antagonist sufficient to reduce or decrease undesirable or abnormal eosinophil migration, chemotaxis or generation thereby treating the subject.

16. The method of claim 15, wherein the condition comprises a chronic or acute allergic disorder.

17. The method of claim 16, wherein the allergic disorder is selected from: Extrinsic bronchial asthma; Allergic rhinitis; Onchocercal dermatitis; Atopic dermatitis, Drug reactions; Nodules, eosinophilia, rheumatism, dermatitis, and swelling (NERDS); Eosophageal and GI allergies.

18. The method of claim 15, wherein the condition comprises a vasculitic granulomatous disease.

19. The method of claim 18, wherein the vasculitic granulomatous disease is selected from: Temporal vasculitis; Churg-Strauss syndrome; Polyarteritis; Wegener's granulomatosis; Eosinophilic granulomatous prostatitis; and Ulercerative colitis.

20. The method of claim 15, wherein the condition comprises an immunological disorder.

21. The method of claim 20, wherein the immunological disorder comprises an autoimmune disease.

22. The method of claim 21, wherein the autoimmune disease comprises multiple sclerosis.

23. The method of claim 20, wherein the immunological disorder is selected from: graft rejection and Intrinsic bronchial asthma.

24. The method of claim 15, wherein the condition comprises an interstitial disorder or a pulmonary disorder.

25. The method of claim 24, wherein the interstitial or pulmonary disorder is selected from: Eosinophilic pleural effusions; Transient pulmonary eosinophilic infiltrates (Löffler); Histiocytosis; Chronic eosinophilic pneumonia; Hypersensitivity pneumonitis; Allergic bronchopulmonary aspergillosis; Sarcoidosis; Idiopathic pulmonary fibrosis; pulmonary edema; pulmonary embolism; pulmonary emphysema; Pulmonary Hyperventilation; Pulmonary Alveolar Proteinosis; Chronic Obstructive Pulmonary Disease; Interstitial Lung Diseases; and Topical eosinophilia.

26. The method of claim 15, wherein the condition comprises a respiratory disorder or a respiratory mucosum disorder.

27. The method of claim 15, wherein the respiratory or respiratory mucosum disorder is selected from: Airway Obstruction, Apnea, Asbestosis , Atelectasis, Berylliosis, Bronchiectasis, Bronchiolitis, Bronchiolitis Obliterans Organizing Pneumonia, Bronchitis, Bronchopulmonary Dysplasia, Common Cold, Cough, Empyema, Pleural Empyema, Pleural Epiglottitis, Hemoptysis, Hypertension, Kartagener Syndrome, Meconium Aspiration, Pleural Effusion, Pleurisy, Pneumonia, Pneumothorax, Respiratory Distress Syndrome, Respiratory Hypersensitivity, Respiratory Tract Infections, Rhinoscleroma, Scimitar Syndrome, Severe Acute Respiratory Syndrome, Silicosis, Tracheal Stenosis and Whooping Cough.

28. The method of claim 15, wherein the condition comprises a neoplastic or myeloproliferative disease.

29. The method of claim 28, wherein the neoplastic or myeloproliferative disease comprises Hypereosinophilic syndrome.

30. The method of claim 15, wherein treatment reduces, decreases, inhibits, delays, eliminates or prevents the probability, severity, frequency, or duration of one or more symptoms associated with or caused by the condition, disorder or disease.

31. A method of reducing or decreasing the probability, severity, frequency, duration or preventing a subject from having an acute asthmatic episode, comprising administering to a subject that has previously experienced an asthmatic episode or has been diagnosed as having asthma with an amount of 5-HT2A receptor antagonist sufficient to reduce or decrease the probability, severity, frequency, duration or prevent an acute asthmatic episode.

32. The method of claim 31, wherein the acute asthmatic episode is caused by allergic asthma.

33. A method of increasing airway-dilation, comprising administering to a subject in need of increasing airway-dilation an amount of 5-HT2A receptor antagonist sufficient to increase airway-dilation in the subject.

34. A method of reducing the probability, severity, frequency, duration or preventing airway-constriction, comprising administering to a subject in need of reducing the probability, severity, frequency, duration or preventing airway-constriction an amount of 5-HT2A receptor antagonist sufficient to reduce or decrease the probability, severity, frequency, duration or prevent airway-constriction in the subject.

35. The method of claim 1, further comprising contacting or administering a second drug to the subject prior to, with or following contacting or administering the 5-HT2A receptor antagonist.

36. The method of claim 35, wherein the second drug comprises an anti-inflammatory, anti-asthmatic or anti-allergy drug.

37. The method of claim 35, wherein the second drug comprises a hormone or a steroid.

38. The method of claim 35, wherein the second drug comprises an anti-histamine, anti-leukotriene, anti-IgE, anti-α4 integrin, anti-β2 integrin, anti-CCR3 antagonist, β2 agonist or anti-selectin.

39. The method of claim 12, wherein the antagonist is selective for 5-HT2A receptor.

40. The method of claim 39, wherein the antagonist comprises a 4-piperidine-methanol or N-aralkyl-piperidine-methanol derivative.

41. The method of claim 39, wherein the antagonist comprises (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer) or prodrug thereof.

42. The method of claim 12, wherein the amount administered is sufficient to reduce or decrease progression, severity, frequency, probability, duration or prevent one or more adverse physiological or psychological symptoms associated with allergic asthma, a disorder associated with undesirable or abnormal eosinophil migration, chemotaxis or generation, or an acute asthmatic episode.

43. The method of claim 12, wherein the subject is a mammal.

44. The method of claim 12, wherein the subject is a human.

45. The method of claim 12, wherein the subject has been diagnosed as having asthma.

46. The method of claim 12, wherein the subject is administered the 5-HT2A receptor antagonist one, two, three, four or more times daily, weekly, monthly or annually.

47. The method of claim 12, wherein the subject has not previously been administered a 4-piperidine-methanol or N-aralkyl-piperidine-methanol derivative; (+)-α-(2,3-dimethoxyphenyl)-1-[2-(4-fluorophenyl)ethyl]-4-piperidinemethanol (MDL-100,907); α-(3,4-dimethoxyphenyl)-1-[2-(4-fluorophenyl)ethyl]-4-piperidine methanol; α-(3,5-dimethoxyphenyl)-1-[2-(4-fluorophenyl)ethyl]-4-piperidine methanol; α-(3,4,5-trimethoxyphenyl)-1-[2-(4-fluorophenyl)ethyl]-4-piperidine methanol; α-(2-methoxyphenyl)-1-[2-(4-fluorophenyl)ethyl]-4-piperidine methanol; α-(2,3-dimethoxyphenyl)-1-[2-{(morpholino)ethyl}]-4-piperidine methanol; α-(2,3-dimethoxyphenyl)-1-[2-(4-trifluoromethylphenyl)ethyl]-4-piperidine methanol; α-(2,3-dimethoxyphenyl)-1-[2-(4-methoxyphenyl)ethyl]-4-piperidine methanol; α-(2,3-dimethoxyphenyl)-1-[2-(4-(2′,2′,2′-trifluoroethoxyphenyl)ethyl]-4-piperidine methanol, or a salt, free base, ester, derivative, racemate (+ or − enantiomer) or prodrug thereof for treatment of an acute or chronic neurological or psychological disorder.

48. The method of claim 47, wherein the neurological or psychological disorder is anxiety, anorexia nervosa, insomnia, sleep apnea, obsessive compulsive disorder, psychosis, bipolar disorder, depression, dysthymia, schiozophrenia, mania, substance abuse, or migraines.

49. The method of claim 48, wherein the psychosis comprises brief, shared or substance-induced delusions, hallucinations, or illusions.

50. The method of claim 48, wherein the substance abuse comprises chronic or acute alcohol, nicotine, a narcotic, an opiate, a stimulant, cocaine, amphetamine, methamphetamine or dextroamphetamine abuse.

51. The method of claim 12, wherein the subject has not previously been administered a 5-HT2A receptor antagonist for treatment of an acute or chronic cardiac disorder, vascular disorder or hypertension.

52. The method of claim 51, wherein the cardiac disorder is myocardial infarction, ischemia, stable or variant angina, coronary vasospasms, or coronary arrhythmia.

53. The method of claim 52, wherein the coronary arrhythmia is atrial tachycardia, atrial flutter, atrial fibrillation, ventricular tachycardia or ventricular fibrillation.

54. The method of claim 51, wherein the vascular disorder is a peripheral vascular disease, glaucoma, intermittent claudication, peripheral vasospasms, a thrombotic illness, an embolitic illness or stroke.

55. The method of claim 12, wherein the subject has not previously been administered a 5-HT2A receptor antagonist for treatment of fibromyalgia or Raynaud's phenomenon.

56. The method of claim 12, wherein the subject has not previously been administered a 5-HT2A receptor antagonist for an anesthetic or analgesic.

57. The method of claim 12, wherein the subject has not previously been administered a 5-HT2A receptor antagonist for treatment of an acute or chronic extrapyramidal side effect (EPSE) associated with a neuroleptic drug.

58. The method of claim 57, wherein the EPSE is dysphoria, akathisia, a cognitive impairment, parkinsonian-like syndrome, tremors, or loss of motivation.

59. The method of claim 12, wherein the amount administered is about 0.00001 mg/kg, to about 10,000 mg/kg, about 0.0001 mg/kg, to about 1000 mg/kg, about 0.001 mg/kg, to about 100 mg/kg, about 0.01 mg/kg, to about 10 mg/kg, about 0.1 mg/kg, to about 1 mg/kg one, two, three, four, or more times per hour, day, week, month or annually.

60. The method of claim 12, wherein the amount administered to the subject is less than about 0.00001 mg/kg, one, two, three, four, or more times per hour, day, week, month or annually.

61. The method of claim 12, wherein the amount is administered to the subject substantially contemporaneously with, or within about 1-60 minutes, hours, or days of the onset of a symptom associated with allergic asthma, an asthmatic episode or airway-constriction.

62. The method of claim 12, wherein the 5-HT2A receptor antagonist is delivered to the lungs or airways.

63. The method of claim 12, wherein the 5-HT2A receptor antagonist does not traverse the blood-brain or blood-spinal cord barrier of the subject in sufficient amounts effective to treat any of the conditions, disorders or diseases set forth in claims 47 to 58.

64-89. (canceled)
